(19) United States
(12) Patent Application Publication
(10) Pub. No.: US 2025/0345164 A1
(43) Pub. Date: Nov. 13, 2025

(54) DIRECT CONTACT TREATMENT OF AQUATIC ANIMALS AND OTHER SUBMERGED TARGET BODIES VIA VISCOUS AND HYDROPHOBIC DELIVERY COMPOSITIONS

(71) Applicant: Ocean Alchemists LLC, Tampa, FL (US)

(72) Inventors: Katie Sybil Curtis, Tampa, FL (US); Michael David Favero, Tampa, FL (US)

(21) Appl. No.: 19/200,635

(22) Filed: May 6, 2025

Related U.S. Application Data
(60) Provisional application No. 63/644,349, filed on May 8, 2024.

(51) Int. Cl. A61D 7/00; A01N 25/24; A01P 1/00; A61K 9/00; A61K 45/06

(52) U.S. Cl. CPC A61D 7/00; A01N 25/24; A01P 1/00; A61K 9/0014; A61K 45/06

(57) ABSTRACT

At least three compositions and a method of using the compositions in a liquid environment on target regions of target bodies such as diseased portions on a colony of coral. At least one base agent having a hydrophobic property provides at least one beneficial characteristic, such as light blocking. In some embodiments, at least one active agent provides a therapeutic property such as an anti-microbial characteristic which is conveyed to the target region during a period of time defined as the diffusion period. In some embodiments, at least one release modifier agent is added to the composition to accelerate or slow down the conveyance of the therapeutic property to the target region. The target region of the target body is located either continuously submerged in a liquid environment or submerged during high tide when the liquid environment is subject to tidal forces. The ingredients are evenly mixed to form a composition body which is applied to the target region of the target body. The compositions are each highly viscous and have an adhesive property.

### RELATED APPLICATIONS

This application relates to and claims priority benefits from U.S. Provisional Patent Application No. 63/644,349, entitled “Direct Contact Treatment Of Aquatic Animals and Other Submerged Target Bodies Via Viscous and Hydrophobic Delivery Compositions,” filed May 8, 2024, which is hereby incorporated by reference in its entirety.

### TECHNICAL FIELD

The present disclosure relates to at least three compositions that provide a direct contact application to aquatic animals, plants or objects. These compositions may be fashioned to deliver or administer a therapeutically effective amount of treatment or provide benefit over a relatively long period of time while being subjected to a liquid environment at least during high tide.

### BACKGROUND

Delivering treatment to aquatic lifeforms such as coral and to submerged non-organic objects such as submerged boat hulls is challenging. Colonies of coral have defensive mechanisms that slough off many applied treatments. Sharks aggressively avoid capture which prevents the delivery of treatment to the animal's wounds and makes the re-application of treatment next to impossible. Applying treatment to underwater man-made objects such as stairs can require multiple repeated treatments. Several aquatic lifeforms, such as colonies of coral, are also not submerged at low tide and so create a more difficult scenario for applying long lasting treatment. Fish can eat away many applied treatments applied underwater. Further, tidal and Oceanic conditions can create currents which erode away any underwater treatment. In short, current strategies to administer treatment to aquatic animals such as diseased colonies of coral require repeated applications and accommodations for the likely loss of most of the treatment to erosion, succumbing to the animal's defensive responses, and the consumption of the treatment by fish and other aquatic wildlife.

Former attempts to administer treatments to diseased colonies of coral and other aquatic animals have been deemed ineffective. The treatments slough off and float away, are not strong enough to work effectively, or are indeed strong enough to work, but then end up delivering too much treatment everywhere except where it is intended. In addition, these prior art treatments place divers at risk excessively due to the need to provide repeated short-term applications.

The compositions recited herein and the method of their use to deliver treatment or provide benefit in an aquatic environment to target regions on target bodies such as diseased portions of colonies of coral over relatively long periods of time are intended to reduce the number of re-applications required, reduce the amount of wasted material, and save the divers time and risk when compared to the former attempts to deliver treatment for long periods of time in an aquatic environment.

### SUMMARY

According to one aspect of the invention, a method of using a composition suitable for use in a liquid environment to convey a therapeutic effect includes providing at least one base agent and at least one active agent. The base agent is provided and has a hydrophobic property and at least one beneficial characteristic. The at least one active agent is provided and has at least one therapeutic property. A target body having a target region is provided. A liquid environment made of at least 75% water is provided. A formulation including the at least base agent and the at least one active agent are mixed together evenly to form a first composition body. The first composition body is applied to the target region for at least a period of time that defines a diffusion period. The first composition body diffuses a therapeutically effective amount of the at least one active agent which conveys the at least one therapeutic property to the target region during the diffusion period. The first composition body has a viscosity between a range of 500,000 to 50,000,000 centipoise, preferably a range of 750,000 to 40,000,000 centipoise, or more preferably a range of 1,000,000 to 30,000,000 centipoise when the viscosity is measured as follows. The viscosity of the first composition body is measured by heating the first composition body to 25 degrees Celsius, using a T Bar Spindle (A-F) with helipath and taking the viscosity reading after rotating the first composition body between 0 rotations per minute to 5 rotations per minute at 1 minute after the cross bar of the spindle enters the first composition body. The first composition body has an adhesive property. The target region is submerged in the liquid environment continuously during the diffusion period or is submerged in the liquid environment at high tide as a result of the liquid environment being subject to tidal forces.

According to one aspect of the invention, the liquid environment comprises at least 80% water, preferably at least 85% water, or more preferably at least 95% water. The first composition body is in contact with the target region during the entire length of the diffusion period. According to another aspect of the invention, the first composition body diffuses a therapeutic effective amount of the at least one active agent to the target region, wherein the diffusion period is at least 12 hours, preferably at least 24 hours, more preferably at least 48 hours, or most preferably at least 72 hours. According to one aspect of the invention, the at least one beneficial characteristic is air impermeability, air permeable retardation, liquid impermeability, liquid permeable retardation, a light diffusing effect, a light blocking effect, a viral impermeability, a viral permeable retardation, a bacterial impermeability, or a bacterial permeable retardation. According to one aspect of the invention the composition further includes at least one taste deterrent characteristic that is configured to discourage aquatic life such as fish from eating the first composition body, at least one antioxidant/preservative, at least one astringent, at least one pigment, at least one efficacy enhancing agent, or at least one adhesive agent.

According to another aspect of the invention, the method of using the composition includes a further step of providing at least one release modifier. The formulation of the first body composition further comprises the at least one release modifier agent in the formulation. The at least one release modifier agent slows or accelerates the conveying of the at least one therapeutic property of the at least one base agent to the target region during the diffusion period.

According to another aspect of the invention, the at least one therapeutic property has an antimicrobial, an antifungal, an antiviral, an antiprotozoal, an antiparasitic, an antiseptic, an anesthetic, a toxic, a nutrient providing, a quorum inhibiting, an algae providing, or an anti-inflammatory characteristic. In one aspect of the invention, the at least one therapeutic property is conveyed to an aquatic organism belonging to Anthozoa, Subphylum Crustacea, superclass Osteichthyes, aquatic mammals, or aquatic reptiles. In one aspect of the invention, the at least one therapeutic property is conveyed to an aquatic organism belonging to Anthozoa, aquatic mammals, or aquatic reptiles. In another aspect of the invention, the first composition body diffuses a therapeutically effective amount of the at least one active agent to the target region, wherein the diffusion period is at least 12 hours, preferably at least 24 hours, more preferably at least 48 hours, or most preferably at least 72 hours. In another aspect of the invention, the diffusion period conveys the at least one therapeutic property to the target region from the at least one active ingredient at a first rate during a first time interval, at a second rate during a second time interval, and at a third rate during a third time interval. The first rate is greater than the second rate when the first time interval is before the second time interval, and the second rate is greater than the third rate when the second time interval is before the third time interval.

According to one aspect of the invention, a method of using a composition suitable for use in a liquid environment that has a beneficial effect includes providing at least one base agent. The at least one base agent has a hydrophobic property and at least one beneficial characteristic. A target body with a target region is provided. A liquid environment made of at least 75% water is provided. A formulation is mixed evenly that comprises the at least one base agent to form a first composition body having an adhesive property. The first composition body is applied to the target region for at least a period of time defining a first interval of time. The first composition body has a high viscosity that is measured as follows. The first composition body has a viscosity between a range of 500,000 to 500,000,000 centipoise, preferably a range of 750,000 to 40,000,000 centipoise, more preferably a range of 1,000,000 to 30,000,000 centipoise, when the viscosity is measured by heating the first composition to 25 degrees Celsius, using a T Bar Spindle (A-F) with helipath and taking the viscosity reading after rotating the first composition between 0 rotations per minute to 5 rotations per minute at 1 minute after the cross bar of the spindle enters the first composition body. The target region is submerged in the liquid environment continuously or is submerged in the liquid environment at high tide as a result of the liquid environment being subject to tidal forces during the first interval of time.

According to another aspect of the invention, the liquid environment comprises at least 80% water, preferably at least 85% water, or more preferably at least 95% water, and wherein the first composition body is in contact with the target region during the entire length of the first interval of time. According to another aspect of the invention, the first interval of time is at least 12 hours, preferably at least 24 hours, more preferably at least 48 hours, or most preferably at least 72 hours. According to another aspect of the invention, the target region is located on a surface of an aquatic organism belonging to Anthozoa, Subphylum Crustacea, superclass Osteichthyes, aquatic mammals, or aquatic reptiles. According to another aspect of the invention, the target region is located on a surface of an aquatic organism belonging to Anthozoa, aquatic mammals, or aquatic reptiles. In another aspect of the invention, the at least one beneficial characteristic is air impermeability, air permeable retardation, liquid impermeability, liquid permeable retardation, a light diffusing effect, a light blocking effect, a viral impermeability, a viral permeable retardation, a bacterial impermeability, or a bacterial permeable retardation. In another aspect of the invention, the at composition further includes at least one taste deterrent that is configured to discourage aquatic life such as fish from eating the first composition body, at least one antioxidant/preservative, at least one astringent, at least one pigment, at least one efficacy enhancing agent, or at least one adhesive agent.

According to one aspect of the invention, a method of using a composition suitable for use in a liquid environment that has a beneficial effect includes providing at least one base agent. The at least one base agent has a hydrophobic property and at least one beneficial characteristic. A target body with a target region is provided. A liquid environment made of at least 75% water is provided. A formulation is mixed evenly that comprises the at least one base agent to form a first composition body having an adhesive property. The first composition body is applied to the target region for at least a period of time defining a first interval of time. The first composition body has a high viscosity that is measured as follows. The first composition body has a viscosity between a range of 500,000 to 500,000,000 centipoise, preferably a range of 750,000 to 40,000,000 centipoise, more preferably a range of 1,000,000 to 30,000,000 centipoise, when the viscosity is measured by heating the first composition to 25 degrees Celsius, using a T Bar Spindle (A-F) with helipath and taking the viscosity reading after rotating the first composition between 0 rotations per minute to 5 rotations per minute at 1 minute after the cross bar of the spindle enters the first composition body. The target region is submerged in the liquid environment continuously or is submerged in the liquid environment at high tide as a result of the liquid environment being subject to tidal forces during the first interval of time. The at least one therapeutic property is conveyed to an inorganic aquatically located substrate such as a set of stairs, a reef module, a shell produced by a barnacle, a statue, a pane of glass, a pane of acrylic, a propeller, an aquarium, or a boat hull.

### BRIEF DESCRIPTION OF THE DRAWINGS

The present disclosure will become more easily understood when reading the following detailed description in conjunction with the accompanying drawings, throughout the drawings, like reference numbers like parts, in the drawings:

FIG. 1A is a reproduction of an image of a colony of coral suffering from Stony Coral Tissue Loss Disease with a diseased or target region and a healthy or first non-targeted region and a dead or second non-targeted region shown;

FIG. 1B is a reproduction of an image of another colony of coral suffering from Black Band Disease with a diseased or target region, a healthy or first non-targeted region, and a dead or second non-targeted region shown;

FIG. 1C is a reproduction of an image of a manatee suffering from a wound in need of treatment;

FIG. 1D is a reproduction of an image of a shark suffering from a wound in need of treatment;

FIG. 2 is an illustration of a representative colony of coral that serves as a target body with a target region that is suffering with Black Band Disease, with the elevations of the liquid environment illustrated at both high tide and low tide;

FIG. 3 illustrates the colony of coral of FIG. 2 with a detailed expanded view provided that better shows the details of a polyp from the colony of coral, and a detailed expanded view provided that better shows the details of the tissue of the polyp;

FIG. 4 illustrates three different compositions bodies: a first composition, a second composition and a third composition which are distinguished from one another by the ingredients that are contained within the mixtures;

FIG. 5 illustrates another representative colony of coral similar to the colony of coral of FIG. 2 that includes a detailed expanded view that shows one healthy polyp of the colony of coral and one unhealthy or dead polyp of the colony of coral, with an application of one of the composition bodies similar to the composition bodies illustrated in FIG. 4 positioned in contact with a first target boundary and a second target boundary of the target region of the target body, namely the colony of coral;

FIG. 6 illustrates another representative colony of coral similar to the colony of coral of FIG. 2 that is positioned submerged in the liquid environment, resting on the seafloor, the colony of coral serving as the target body with the diseased region of the colony of coral serving as the designated target region, with a direct application and an indirect application of two composition bodies similar to those shown in FIG. 4, to the target region shown with both compositions illustrated in cross section;

FIG. 7 illustrates another representative composition body similar to one of the composition bodies shown in FIG. 4, in three different stages of use, with the composition body displaying a first color at a first time interval, displaying a second color at a third time interval, and displaying a combination of the first color and the second color at a second time interval;

FIG. 8 illustrates an exemplary colony of bacteria forming a biofilm with exposure to an exemplary composition body similar to composition bodies shown in FIG. 4, and the sloughing off of the composition body as a defensive response over several time intervals;

FIG. 9 illustrates another exemplary colony of bacteria attempting to form a mucosal response with exposure to another exemplary composition body similar to the one of the composition bodies shown in FIG. 4 that includes at least one quorum inhibiting agent or at least one astringent, and the impact on the sloughing off defensive response of the colony of bacteria over several time intervals;

FIG. 10 is a reproduction of several images that represent a variety of exemplary organic and non-organic target bodies;

FIG. 11 is a reproduction of a graph that measures time on one axis, and the accumulated dispensation or diffusion of the at least one active agent from two exemplary composition bodies during a diffusion period;

FIG. 12 is a series of illustrations that collectively depict the change of an absorbent fiber or absorbent medium into a woven sheet of absorbent fiber or medium that is impregnated and/or coated with a representative composition body.

### DETAILED DESCRIPTION

This disclosure will be broken into two informal sections. The first section will be a more general conversation about the invention that may reference the FIGS., but does not describe the drawings in great detail. In the second section, each of the FIGS. is addressed in careful detail to avoid any potential confusion about the scope and bounds of the claimed invention.

Corals are marine invertebrates from the phylum Cnidaria which also includes jellyfish, anemone, & hydroids. Corals as the general public tends to understand them are actual colonies of coral comprised of individual animals known as coral polyps. These individual polyps filter feed from the surrounding seawater using tentacles and a mouth. Hard corals even have shared tissue between polyps known as the coenosarc which allows them to distribute nutrients throughout the entire coral colony as needed. Beneath the fleshy coral tissue lies a skeleton of calcium carbonate that is created by the coral's excrement and is shared among the colony. These continuously growing skeletons are the building blocks of coral reefs and are the reason why corals are so valuable to the ocean's ecosystems.

Like all animals, corals are susceptible to a variety of ailments including viruses, infections and other diseases. These ailments are often influenced by environmental factors and can be bacterial, viral, fungal, or otherwise influential to the corals' ability to obtain essential nutrients and/or requirements for healthy functioning. Once a coral experiences one form of ailment they become immunocompromised and are therefore susceptible to further infection. However, there are very few treatment options available for corals who become ailed with disease or infection. Some of these ailments include but are not limited to, black band disease (BBD), white band disease (WBD), white plague, stony coral tissue loss disease (SCTLD), brown jelly disease (BJD), and brown ciliates.

For many coral diseases, the ailment will progress across the coral colony attacking neighboring polyps which can result in tissue loss, exposing the skeleton beneath. This area is known as the disease margin where there is a visible division between healthy tissue and the exposed skeleton with the diseased margin lying in-between. The tissue loss often progresses in speeds and patterns that can be indicative of the specific disease and can often play a role in disease identification. The disease margin, the “lesion line” or “target boundary” is also generally the most effective area to target treatment of these diseases/infections as it normally contains the majority of the ailing organisms and has minimum impact on the remaining healthy polyps.

Corals also share a symbiotic relationship with a photosynthetic organism collectively known as zooxanthellae, a microscopic algae, which live in the tissue of the coral polyps and provide the corals with nutrients obtained from photosynthesis in exchange for shelter within the coral colony. Zooxanthellae also contribute to the color that most people associate with coral. These zooxanthellae require specific temperatures and amounts of light to function which in turn impacts the health of the coral host. For example, if temperatures get too high, the coral will expel its zooxanthellae giving the tissue a white appearance and will enter a vulnerable state of minimized metabolism known as coral bleaching. During coral bleaching, the coral polyps are not receiving the proper nutrients and are therefore particularly susceptible to other ailments such as coral disease and viral or fungal infections.

Other aquatic creatures such as manatees and sea turtles are susceptible to viruses and disease which can cause lesions on the tissue which are historically left untreated. Manatees in particular are often struck by boat propellers which leave large gashes in their flesh which become prone to infection. Sharks, rays and other aquatic animals often receive cuts from bumping into the reef or from battling with their prey or other predators. These wounds can be fatal if left untreated.

The proposed invention seeks to treat aquatic animals such as but not limited to coral, fish, sea turtles, sharks, reptiles, amphibians, manatees, whales, and rays utilizing a direct contact or indirect contact application of a diffusive hydrophobic composition body that can be extruded from either a caulking-type gun, a syringe, a sausage-type casing, or even shaped by hand to be adhered directly onto or near the target region of the target body. This method of treatment allows only the infected part of the animal to receive the majority of the treatment and thus minimizes any potential impact to the healthy tissue of the organism.

Providing a long-term hydrophobic adhesive diffusive composition body embedded with active agents to supply treatment to a target body without the need to re-apply provides many advantages when providing treatment to an aquatic organism or object. The proposed invention can provide a means to topically treat ailing organisms that would otherwise require exhaustive application measures like daily scuba dives or daily organism capture and releases to continue supplying treatment.

The proposed invention utilizes formulation expertise to create unique viscous, hydrophobic composition bodies designed to adhere near or directly onto a submerged target region while providing in at least one embodiment sustained release of one or more active agents by means of diffusion to provide a one-time direct contact application full treatment for a minimum of 12 hours without the need to reapply. The utilization of a logarithmic diffusion profile which diffuses a minimum of 25% by weight, preferably 40% by weight of available active agent ensures that the target region receives an initial large therapeutic dose followed by gradually decreasing “maintenance” doses which, in at least one embodiment, prevents reinfection without the concern of over treating. In at least one embodiment the use of release agent modifiers impacts the active agents' ability to be diffused from the composition body in a useful way. In at least one embodiment, the invention is intended for use on non-human species and objects, specifically aquatic lifeforms and submerged man-made objects.

Therefore, the treatment provided from a single application for a minimum of 12 hours without the need to reapply ensures that stress is minimized on the subject by limiting the number of capture and releases required for full treatment of the targeted species while simultaneously limiting administrator safety concerns. A 12 hour minimum and/or one-time treatment of the described mechanism becomes even more valuable when some ailing organisms are near impossible to recapture or would require extreme risk to do so. Examples of this include treating wounds on fish capable of finding refuge in a coral substrate or shark wounds, where the stress of capture of the shark often results in death.

Treatments for human subjects rarely utilize a sustained diffusive profile greater than 12 hours. Presumably, this is due to the relative ease associated with providing additional or follow-up dosing to human subjects. In contrast to this, active agent redosing or application to nonhuman species, especially but not limited to those of aquatic origin, is often difficult, costly, and dangerous to both the administrator and ailing organism.

Therefore, there is a need to create a composition capable of a “one dose” application which continues to adhere and deliver active agent(s) to the site of an ailing organism throughout the lifespan of the required treatment.

The ability of the composition body to adhere directly to the tissue or target region of the target body is a very valuable feature in many of the contemplated embodiments. Applications are either performed underwater, applied to target bodies that will be submerged at high tide, or applied onto animals above water which are then reintroduced into an aqueous environment shortly thereafter. The ability to create the proper adhesion for application is a very valuable feature that helps ensure that the composition body remains in place long enough to successfully deliver the treatment.

The high viscosity of the proposed compositions is a valuable feature that helps to assure that the proposed compositions remain secured to the desired positions to deliver the treatment or benefit. Specifically, a viscosity between a range of 500,000 centipoise to 50,000,000 centipoise measured at a temperature of 25° C. was found to yield the best results after an exhaustive amount of experimentation. This viscosity range is higher than is commonly utilized for most topical applications and was discovered by the inventors to be very effective for the composition's intended use. The range allows the product to be extruded from a variety of dispensers but remain capable of retaining the dispensed shape. This shape holding ability allows the formulation or composition to be pushed into crevices and better contact textured and contoured surfaces, effectively anchoring the composition into a fixed dispensing or diffusing position. When this selected shape forming viscoelasticity is paired with adhesive ingredients, a strong bond to the targeted body may be formed.

Colonies of coral often initiate a mucosal stress response to respond to contact with any foreign body. In response, astringents can be added to the chosen composition to reduce the likelihood of the composition being removed by the coral's mucosal stress response or when bacteria in the localized area have a mucosal stress response. When coral tissue comes in contact with a foreign entity, it often creates an excess of mucous to assist in removing it. Once an excess of mucous is built up, the foreign substance can be sloughed off along with the excess mucous, in this case the ointment or composition, into the surrounding environment. This response can drastically reduce the amount of time that the treating ointment or composition is in contact with the tissue and can thus reduce the effectiveness of the treatment. Astringent compounds bind to proteins in the mucous and thus reduce its ability to retain water and slough off the foreign ointment treatment. This allows the ointment or composition a longer contact time with the coral tissue and thus increases its treatment period.

Since some of the available active agents could be eaten by fish and other marine animals prior to full dispensation, another additional ingredient often added to the composition is at least one taste deterrent. The taste deterrent reduces the likelihood of fish or other aquatic animals eating the composition or ointment and removing the composition from the ailing site before the treatment has been fully released.

Additional ingredients such as efficacy enhancing agents can be added to increase the effectiveness of the active agents. Finally, other additional ingredients such as antioxidants/preservatives can be used to stabilize the active compounds within the composition body to reduce the possibility of degradation and to increase the shelf life of the ointment or composition. Alternatively, the antioxidants/preservatives reduce the growth of unwanted bacteria from developing in the composition while in storage. This allows the composition to be at peak effectiveness when it is applied.

Active agents can include any compounds that can provide or deliver an antiviral, antiseptic, antiprotozoal, antibiotic, antibacterial, antimicrobial, antiparasitic, anesthetic, toxic, or anti-inflammatory characteristic to the composition. Active agent ingredients preferably come from natural sources that, in at least one embodiment, may be effective against the targeted infection/disease. These compounds can be adjusted as needed in order to create the best treatment for the infection/disease that is being treated.

Colonies of bacteria often grow on the surface of targeted bodies, such as colonies of coral. The bacterial response of forming biofilms in response to foreign bodies can interfere with active agent delivery. So, along with antimicrobial agents, quorum inhibiting ingredients can also be added to the composition, as an additional ingredient or alternatively as integrally formed with the at least one active agent. These substances interrupt bacterial communication that result in a wide range of effects. Most notably, quorum inhibiting agents limit the orderly assembly of bacterial cell structuring which otherwise results in a biofilm.

Other nutrients or even a replacement of the symbionts can be added to the composition body to help maintain coral health or to assist coral's survival chances during bleaching events. For example, the active agent in the composition may include nutrients to provide another source of food to the colony of coral during a bleaching event. In addition, or in the alternative, the composition may include a variety of algae such as zooxanthellae in order to assist re-populating the bleached coral with the valuable symbiotic algae.

A valuable feature of several of the compositions is the ability to provide a controlled release of the active agent(s) to the target body over an extended period of time. The treatment is designed to provide an immediate burst of active agent(s) followed by a lower maintenance dosing to ensure the treatment continues to treat the target region of the target body for a longer period of time. The maintenance dosing is customizable, capable of linear to logarithmic release in time frames between as low as 12 hours and up to one month or more.

The dosing technique of an immediate burst followed by maintenance dosing utilizes the physics of diffusion principles to its advantage. Active agent(s) closer to the center of the applied formulation have a longer, more torturous path resulting in slower diffusion. Diffusive pathways may be manipulated by using release modifying agents to allow for active agents to be released from the composition body faster or slower depending on the specific treatment needs.

Due to the dynamic temperature, tides, and currents inherent and unique to each application site, active agent diffusion is prone to alteration and variation in natural environments. To provide scientists and practitioners with a better understanding of how the treatment is proceeding, a visual aide may be applied. In aquatic environments the composition body may be equipped with a pigment or dye to exhibit visible wavelengths below 600 nm. The wavelengths of light exhibited by the dye are preferably in the range of 380 nanometers (nm) to 590 nm, more preferably 380 nm to 520 nm, even more preferably 380 nm to 445 nm. These wavelengths have been chosen as they are highly visible even at great water depths. Color is gradually lost in relation to water depth with longer wavelength colors lost first. The dye diffuses out from the treatment composition body in a manner that is analogous to the active agent(s).

To further elaborate, the dye slowly becomes exhausted as the active agent(s) becomes exhausted. As the at least one pigment diffuses from the composition body, the composition body changes from a first color to a second color such that a display of the first color or the second color by the composition body is an indication of a percentage amount by weight of the at least one active agent that remains within the composition body. This method provides a qualitative visual method of the active agent diffusive rate. The addition of a dye to the composition thereby provides a new, powerful tool for researchers and administrators to document a treatment's progression.

The proposed invention can succeed both in terms of providing long-term adherence and providing active agent delivery topically by means of diffusion effectively creating a “one dose” application lasting a minimum of 12 hours without the need to reapply.

This treatment can contain, but is not required to contain additional ingredients such as adhesives, astringents, antimicrobial agents, taste deterrents, antioxidants/preservatives and efficacy enhancing agents to increase the treatment's overall effectiveness.

In some embodiments, the application of the composition or ointment can be dispensed out of a syringe, a caulking-type tube or a sausage-type casing using a caulk gun or shaped by hand. In at least one embodiment, this creates a cylindrical extrusion of ointment or composition that can be applied directly onto the target boundary of the disease.

The viscoelastic behavior of the composition is a very important feature of the composition. It is beneficial if the composition is capable of application by means of constant repeatable extrusion to apply a uniform bead of material possessing extreme plasticity and capable of significant distortion which can be applied as an unbroken bead of treatment across a variable surface of peaks and crevices present in aquatic animals such as corals. Additionally, the unique flow characteristics of the aquatic environment results in the need for the applied material to be capable of maintaining shape without succumbing to various flow and subsequent sheering effects of currents and waves.

To create a treatment capable of providing a longer period of treatment in an aquatic environment, an uncommonly high viscosity range was required. In contrast, most consumer materials including creams, pastes, hydrogels, and ointments do not target viscosity values above 250,000 centipoise regardless of the selected viscosity testing method. It is conjectured that this is because the energy or force required to dispense materials from their packaging generally exceeds the average humans' capabilities as the viscosity climbs above the viscosity range of 100,000 centipoise. At these large viscosity values, external mechanical forces are often required, and can be achieved conveniently only through the use of syringes, caulking type dispensers, and other dispensing devices.

In order to reliably and consistently measure the viscosity of the compositions in the inventions, a specific and reliable measuring technique was selected. The method utilized requires the use of a helipath in combination with T-bar spindles T-A through T-F which correspond to Brookfield viscometer entry codes 91-96. Measuring at 25° Celsius using the helipath combined with a T-bar spindle rotating in a range between 0 rpm-5 rpm ensures unsheared material is constantly measured as the device apparatus helically rotates down, through the material. By constantly measuring unsheared material the viscoelastic behavior stays constant over time, the method is not subject to dynamic changes that may be a result of additional inherent characteristics of the material such as Thixotropic, Rheopectic, or Elastic behaviors.

The overall compositions described throughout this disclosure differ significantly from hydrogels. Hydrogels are comprised of a three-dimensional network of water swollen, cross-linked polymer chains that absorb large amounts of water which results in increased viscosity. In contrast, each of the overall disclosed embodiments would be more appropriately described as a wax, ointment, salve, silicones, putty, or a dough due to their composition of ingredients which differ from those present in a hydrogel.

A desired target use of this invention is the Florida Reef Tract which extends from Martin County down through the Florida Keys. Depending on the specific location, treatment depth, and season, the water temperatures may change as much as 10° Celsius with currents and wave action varying more. In response to this, the treatment material must also be dynamic which often requires viscosity modifications to meet the dynamic conditions at each site. Due to this, a viscosity range of 500,000 centipoise to 50,000,000 centipoise specifically in correlation with the viscosity method mentioned above, is targeted. This range ensures the treatment material will be capable of proper extrusion from its packaging and subsequent application throughout the many challenging environments its intended to be utilized within.

To clarify the method of measuring the viscosity that leads to the desired above aforementioned viscosity range, the Spindle Code is T-A Through T-F (Entry code 91-96), the speed of the spindle is 0 rpm to 5 rpms, the temperature of the composition being measured is kept at 25° Celsius, and the measurement of the viscosity of the composition is taken one minute after the spindle cross bar enters the composition. Again, the desired broadest range of viscosity, as measured using the above recited method, is between a range of 500.00 Centipoise and 50,000,000 Centipoise, this range is designed to allow material modification to ensure best material extrusion and subsequent adhesion across all tropical regions, all seasons, and water conditions coral resides within. The desired viscosity is more preferably between a range of 750,000 Centipoise-40,000,000 Centipoise, this range is designed to allow material modification to ensure best material extrusion and subsequent adhesion across most tropical regions, during warm water seasons, in all boating conditions. The desired range of viscosity is even more preferably between a range of 1,000,000 Centipoise and 30,000,000 Centipoise. This range is designed to allow material modification to ensure best material extrusion and subsequent adhesion across some tropical regions, during warm water seasons, in favorable boating or aquatic conditions.

In some embodiments the active agent is not required in order for an ointment or composition to provide a beneficial effect. Many bacterial and diseases require light or oxygen in order to proliferate. A topical application of a composition or ointment can create a barrier against light or oxygen to kill harmful bacterial, viruses, or fungal infections. For instance, Black Band Disease, a bacterial infection comprised of cyanobacteria, can be treated by placing an ointment or composition over the disease to prevent light and oxygen penetration. In another instance, the ointment or composition could be applied to cover a wound on a manatee to prevent additional water borne infection by selecting a composition that is sufficiently water insoluble. Topical ointments or compositions can also create a barrier against water borne pathogens by being water insoluble that would otherwise infect the target body.

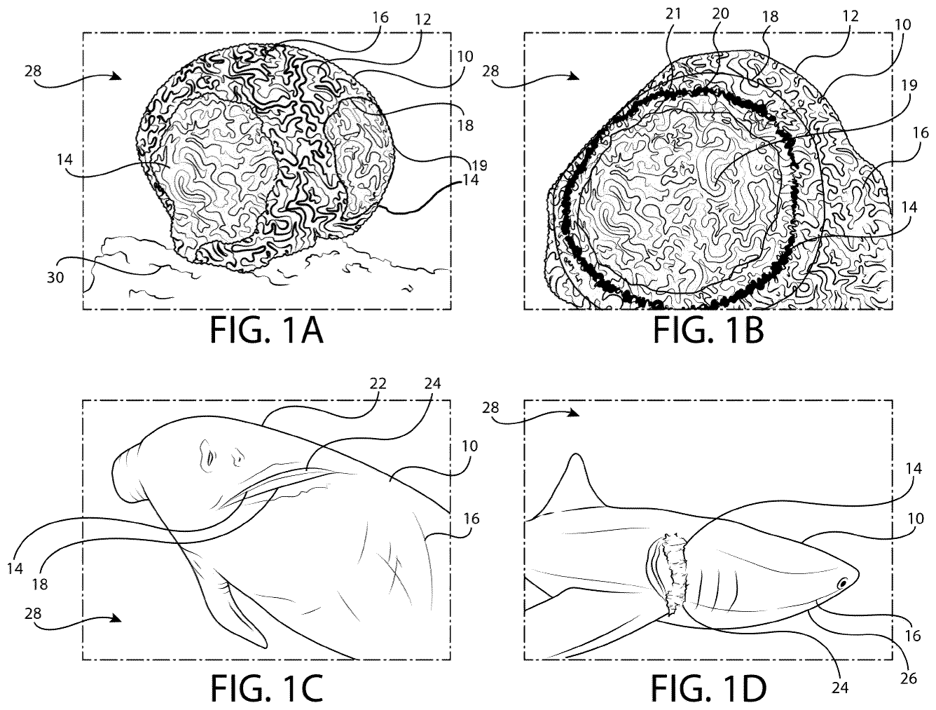

FIGS. 1A, 1B, 1C, and 1D are all reproduced images of animals that are present in an aquatic environment, where applying treatments is difficult. Each of the animals is an example of a body that can be targeted for an application of a composition of treatment to help heal the animal. In FIG. 1A, a target body 10 of a colony of coral 12 has a diseased portion that exhibits signs of stony coral tissue loss disease (SCTLD) that defines one or more diseased or target regions 14 which would benefit from an application of treatment, and one or more healthy or first non-targeted regions 16 which would not benefit from an application of treatment. Often, the first non-targeted regions 16 are healthy, non-diseased portions of the target body 10. However, the first non-targeted regions 16 can be more broadly defined as simply a region that would not benefit from the application of a composition as disclosed below. The compositions below are used for more than just treating diseases.

Referring to FIG. 1A, the target body 10, the target regions 14, and the first non-targeted regions 16 are all three-dimensional bodies with a height, a length, and a depth. The boundary or boundaries between the target regions 14 and the first non-targeted regions 16 are defined as one or more first target boundaries 18. The target boundaries 18 define the division between the target regions 14 and the first non-targeted regions 16, and are two-dimensional surfaces. This means that if the target body 10 was depicted in an exploded view with the volumes of the target regions 14 and the first non-targeted regions 16 shown separated from one another, the one or more surfaces that form the boundary between the target regions 14 and the first non-targeted regions 16 is defined as the one or more first target boundaries 18. This relationship between target bodies 10, target regions 14, first non-targeted regions 16, and first target boundaries 18 does not simply apply to colonies of coral, but to all potential target bodies 10. However, with respect to colonies of coral 12, in some embodiments the target body 10 has a region of dead or unhealthy tissue, which is often referred to as the “exposed skeleton”. This exposed skeleton is identified as a second non-targeted region 19.

FIG. 1B reproduces an image of another exemplary colony of coral 12 that forms another target body 10 that appears to be suffering from another very common coral colony illness, Black Band Disease (BBD). The target body 10 is separated between the target region 14 that exhibits symptoms of BBD, the healthy first non-targeted region 16 that does not exhibit symptoms of BBD, and the dead or second non-targeted region 19. BBD has a distinctive band, often described as a black band 20 that is visible near the first target boundary 18. The black band 20 is positioned between the first target boundary 18 and a second target boundary 21 that separates the second non-targeted region 19 from the target region 14. To be clear, the black band 20 is a symptom of BBD, and represents a portion of the colony of coral 12 that could benefit from an application of treatment. In other words, the target body 10 of the colony of coral 12 can be separated into three regions: a healthy or first non-targeted region 16, a diseased or target region 14, and a dead or second non-targeted region 19. The second non-targeted region 19 is the region of the colony of coral 12 that is now dead or substantially free from live coral polyps because the disease killed the coral polyps. Not all diseased colonies of coral 12 include a dead or second non-targeted region 19, but it is a very common in colonies of coral subject to BBD and other diseases such as SCTLD.

FIG. 1C reproduces an image of an aquatic mammal with an injury that would benefit from treatment, namely a manatee 22 with a large wound 24. In this example, the manatee 22 is the target body 10, the large wound 24 is the target region 14, the healthy non-injured portions of the manatee 22 are the first non-targeted regions 16, and the boundary between the target region 14 and the first non-targeted regions 16 define the first target boundary 18.

FIG. 1D reproduces an image of an Oceanic or saltwater animal with an injury that would benefit from treatment, namely a shark 26 with another exemplary large wound 24. In this example, the shark 26 is the target body 10, the large wound 24 is the target region 14, the healthy non-injured portions of the shark 26 are the first non-targeted regions 16, and the boundary between the target region 14 and the first non-targeted regions 16 define the first target boundary 18.

The respective target bodies 10 of FIGS. 1A-1D are all depicted as being submerged within a respective liquid environment 28. The liquid environment 28 of FIGS. 1A, 1B, and 1D is an Ocean filled with salt water. However, the liquid environment 28 of this disclosure is not limited to that specific embodiment of the Ocean. The liquid environment 28 of FIG. 1C is freshwater. Throughout this disclosure, the liquid environment 28 can be fresh water or salt water. This disclosure embraces and adopts the general demarcation between fresh water and salt water that when a body of water has 35 parts per thousand of salt or salt ions or more it is considered “saltwater”, and when the body of water has less than 35 parts per thousand of salt or salt ions it is considered “freshwater”.

The liquid environment 28 can be an ocean, a lake, a pond, or a more restricted or manmade body of water such as a pool or an aquarium tank. To be clear, an aquarium tank can refer to a small aquarium tank such as one bought at pet stores to house small fish or can be very large aquariums such as ones that are suitable to house whales. The liquid environment 28 can be so large and so deep that the elevation of the liquid environment 28 can discernably change as a result of tidal forces, or could be a shallow or small enough volume that the elevation of the liquid environment 28 does not discernably rise or fall as a result of tidal forces. The liquid environment 28 throughout this disclosure comprises at least 75% by weight of water, preferably at least 80% water, more preferably at least 85% water, or even more preferably at least 95% water. The presence or absence of salt in the liquid environment 28 is not a limiting factor when determining whether the liquid environment 28 is present. The presence of the liquid environment 28 during the application of the ointment or composition enhances the value of the disclosed invention because applying treatments or other equally as important active agents to the target region 14 of the selected target body 10 is much more challenging in a liquid environment 28 than when outside of the liquid environment 28, as discussed above and as will be discussed further below.

The image of FIG. 1A further depicts a wall or floor 30 to the liquid environment 28, often referred to as a “seafloor”. The liquid environment 28, whether an Ocean, a lake, a river, a pool, an aquarium all are bounded or contacted by their respective floors 30, which contributes to defining the boundary of the respective liquid environment 28. This means that the floor or walls of an aquarium, or the cliff walls and the sea floor of an ocean can be described herein as the floor 30 with respect to the accompanying liquid environment 28. In some embodiments, the boundary between the floor 30 and the liquid environment 28 and the floor 30 is very clear: the liquid environment 28 comprises 75% by weight or more of water, and the floor 30 has no water present at all. However, in certain embodiments, mud may have some water present, but simply less than 75% by weight of water in a given volume. Following the convention embraced herein, this would mean that mud may be considered as the wall or floor 30 that borders the associated liquid environment 28.

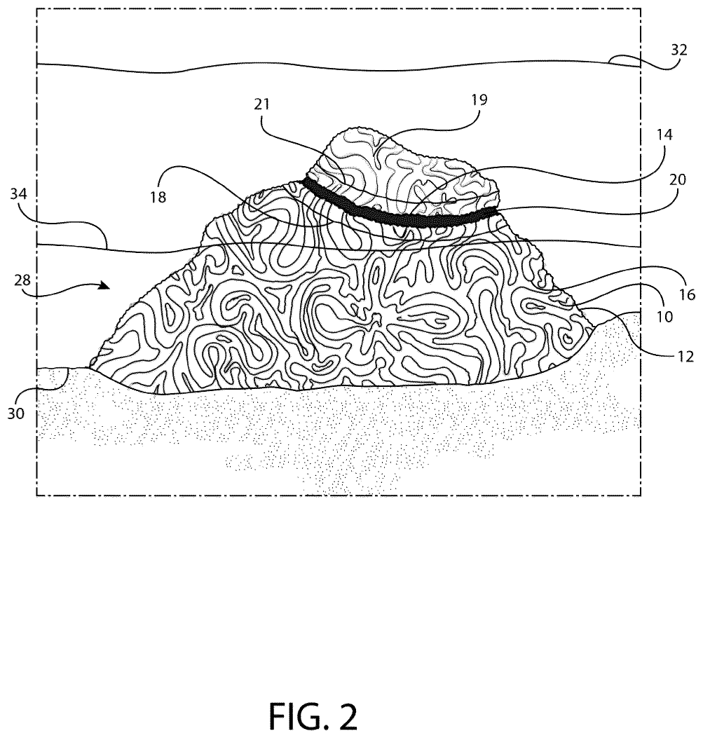

Referring to FIG. 2, another exemplary colony of coral 12 that is an example of another target body 10 shown with the target region 14 for treatment and the first non-targeted region 16 divided by the first target boundary 18 near the black band 20 and at least one second non-targeted region 19 divided from the target region 14 by the second target boundary 21. The liquid environment 28 is bounded by the floor 30. In addition, the elevation of the liquid environment 28 is depicted at both high tide 32 and also at low tide 34. In large bodies of water, such as the Ocean, the target region 14 of various target bodies 10 may be positioned above the liquid environment 28 at low tide 34 and be submerged within the liquid environment 28 at high tide 32. Since colonies of coral 12 are often thriving along the coast of various Oceans, it follows that often times the target regions 14 of target bodies 10 such as colonies of coral 12 will only be submerged in the liquid environment 28 for a portion of every day due to tidal forces. This daily partial exposure of the compositions disclosed herein to the air is another factor that is taken into consideration when evaluating the efficacy of the composition that is to be applied to the various target regions 14. Since in many important embodiments and applications the composition is exposed to the air for a period of time each day, and then submerged in the liquid environment 28 on a daily basis, the compositions disclosed must be resilient and versatile enough to handle these predictable but changing conditions.

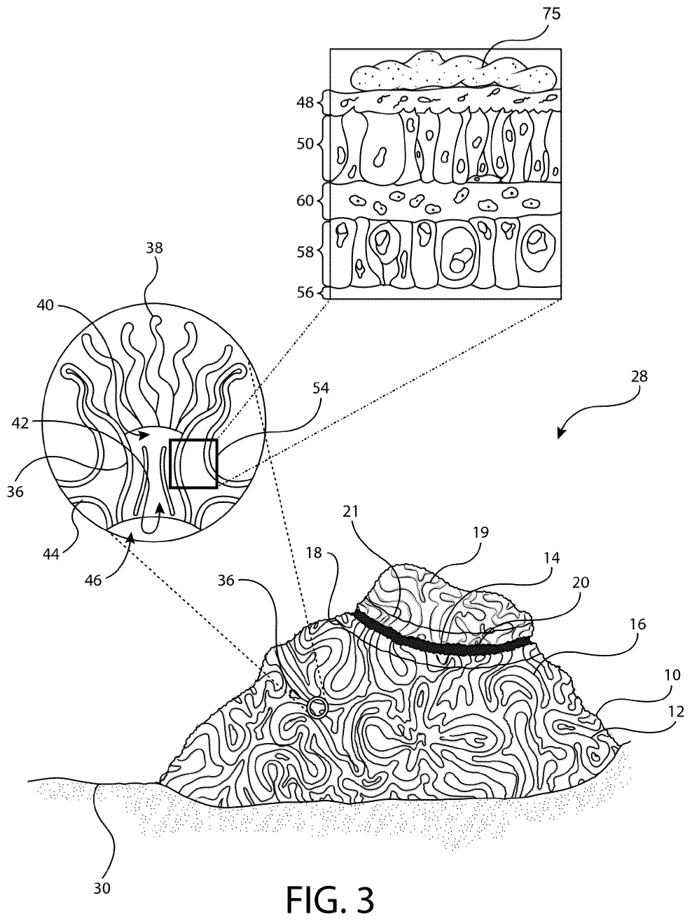

FIG. 3 illustrates another exemplary colony of coral 12, a marine invertebrate, that serves as a potential target body 10 with at least one target region 14, at least one first non-targeted region 16 divided from the target region 14 by the first target boundary 18 located near the black band 20, and at least one second non-targeted region 19 divided from the target region 14 by the second target boundary 21. A portion of the first non-targeted region 16 that magnifies the details of a coral polyp 36 which is part of the colony of coral 12 is shown. The polyp 36 includes one or more tentacles 38, a mouth 40, a stomach 42, a coenosarc 44, and a skeleton 46. The colony of coral 12 is a collection of animals organized as polyps 36 that cooperate with one another to distribute nutrition to one another via the coenosarc 44, collect food from the surrounding liquid environment 28 utilizing their tentacles 38 which carry the captured food to the mouth 40 of the respective polyp 36 to be digested in the stomach 42 of the polyp 36. In addition, polyps 36 often provide a home for photosynthetic organisms known as zooxanthellae (not shown), a microscopic algae, which form a symbiotic relationship with the polyps 36 of the colony of coral 12. The photosynthetic algae (not shown) provide additional nutrition to the polyp 36, and the polyp 36 provides a safe place for the algae to grow and thrive.

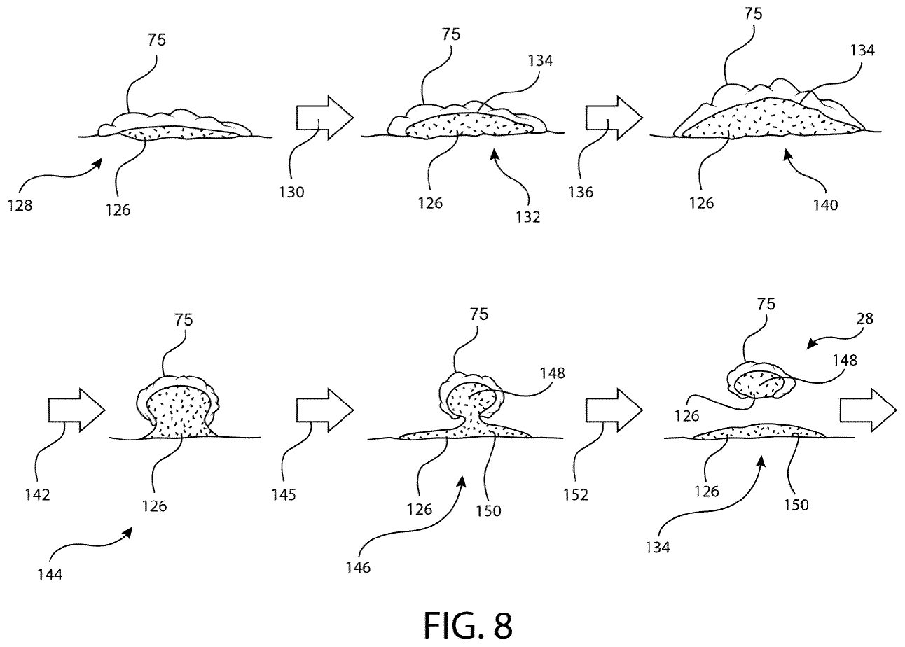

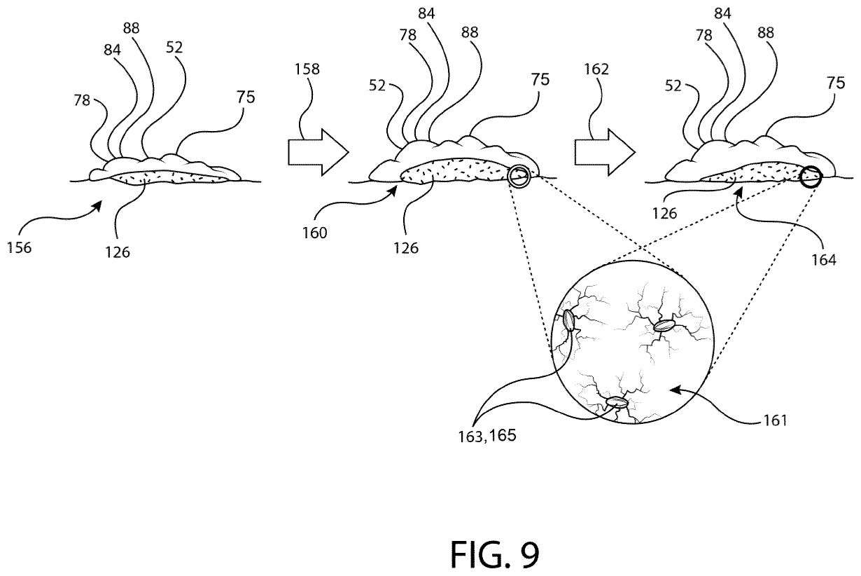

Polyps 36 produce more of the skeleton 46 of the colony of coral 12 as their food is digested. The skeleton 46 is made of mostly calcium carbonate and is very hard and rigid. This skeleton 46 eventually creates the topographical shape that most human observers think of as the shape of coral reefs. It is noteworthy that the zooxanthellae can die or can be expelled by the polyps 36 during times of environmental stress, which leads to the phenomena of “coral bleaching” where the coral turns white or translucent. Both bacteria present on the surface of the coral, and the coral polyps can produce a mucous layer 48 which is created to cover and protect the epidermis layer 50 of the polyp 36. As foreign bodies contact the epidermis layer 50 or the mucous layer 48, the mucous layer 48 tends to remove the foreign bodies by sloughing them off. See the discussion of FIGS. 8 and 9 below for a more detailed explanation.

Although applying the compositions disclosed herein to colonies of coral 12 is clearly a very important application, the compositions are not limited to only being applied to colonies of coral 12. The compositions disclosed herein also serve as a valuable way to deliver important therapeutic effects to other target bodies 10 such as aquatic animals such as fish, sharks 26, manatees 22, and shellfish. But the compositions disclosed herein can also be applied to different target bodies 10 such as kelp, manmade stairs located in the liquid environment 28, reef modules, propellers, and hulls of ships. What all of these various target bodies 10 have in common is that the liquid environment 28, whether the target body 10 remains fully submerged at low tide 34 or is only submerged at high tide 32, make it difficult to dispense an active agent 52 that can convey a therapeutic effect to the target regions 14 of the target body 10 because the liquid environment 28 tends to wash the compositions, along with their active agent(s), away before they can work effectively.

Still referring to FIG. 3, a cross-section of a tissue 54 of the polyp 36 is further magnified to illustrate more details of the coral polyp 36. Layers of the tissue 54 includes a gastro-cavity 56, a gastro-dermis 58, a mesoglea 60, an epidermis layer 50, and a mucous layer 48. The gastro-dermis 58 borders the gastro-cavity 56 where food collected by the tentacles 38 from the surrounding liquid environment 28 is digested. The mesoglea 60 is a layer of cells located between the epidermis layer 50 and the gastro-dermis 58. The epidermis layer 50 is the layer of cells that borders the surrounding liquid environment 28 and the epidermis layer 50 excretes or produces the mucous layer 48 which helps protect the polyps 36 of the colony of coral 12 from foreign bodies.

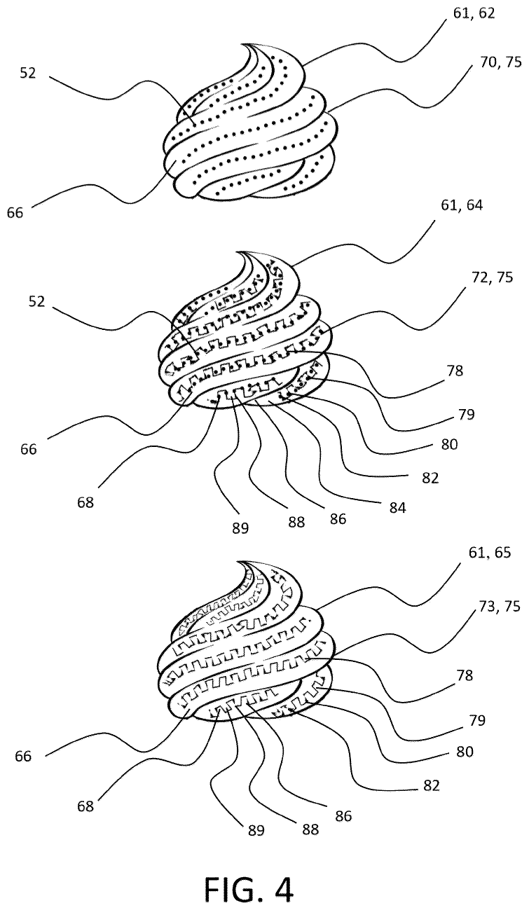

FIG. 4 illustrates three variations or embodiments of a formulation 61 referred to as a first formula 62, a second formula 64, or a third formula 65 respectively. When the first formula 62, the second formula 64 and the third formula 65 are being addressed together, they will consistently and collectively be referred to as the formulation 61. The first formula 62 comprises at least two ingredients, namely at least one base agent 66, and at least one active agent 52. The at least one base agent 66 has a hydrophobic property, and the at least one active agent 52 has at least one therapeutic property. The second formula 64 comprises at least three ingredients, namely at least one base agent 66 which again has a hydrophobic property, at least one active agent 52 which again has at least one therapeutic property, and also at least one release modifier agent 68. The third formula 65 comprises at least two ingredients, namely at least one base agent 66 and at least one additional ingredient 78. When the ingredients of the formulation 61 are mixed together they form their respective formulas 62, 64, 65. The required ingredients are evenly mixed to form a homogenous mixture where the two or three required ingredients that are evenly distributed, the formulation 61 forms a composition body 75. A first composition 70, a second composition 72, or a third composition 73 are formed from the respective formulas 62, 64 and 65. When the first composition 70, the second composition 72 and the third composition 73 are being addressed together, they will consistently and collectively be referred to as the composition body 75. Further, the term composition body 75 can refer to any one of the first, second, or third compositions 70, 72 and 73.

When the first formula 62 is fully mixed so that the ingredients are distributed evenly throughout the mixture of the first formula 62, the first formula 62 forms a corresponding first composition 70. When the second formula 64 is fully mixed so that the ingredients are distributed evenly throughout the mixture of the second formula 64, the second formula 64 forms a corresponding second composition 72. When the third formula 65 is fully mixed so that the ingredients are distributed evenly throughout the mixture of the third formula 65, the third formula 65 forms a corresponding third composition 73. The first composition 70, the second composition 72, and the third composition 73, collectively referred to as the composition body 75, all exhibit an adhesive property defined in more detail below.

The at least one base agent 66 provides several functions to the respective composition body 75. First the at least one base agent 66 forms a physical barrier that provides a beneficial characteristic to the respective composition body 75 such as air impermeability, air permeable retardation, liquid impermeability, liquid permeable retardation, a light diffusing effect, a light blocking effect, a viral impermeability, a viral permeable retardation, a bacterial impermeability, or a bacterial permeable retardation. The base agent also provides a hydrophobic property to the respective composition body 75 which serves to provide a physical barrier from the liquid environment 28 which protects the at least one active agent 52 (if applicable) from washing away before delivering or administering the desired therapeutic effect in the liquid environment 28 to the target region 14 of the designated target body 10. The at least one base agent 66 can contribute to creating an adhesive property for the respective composition body 75. However, additional ingredients can also help create the desired adhesive property for the respective composition body 75.

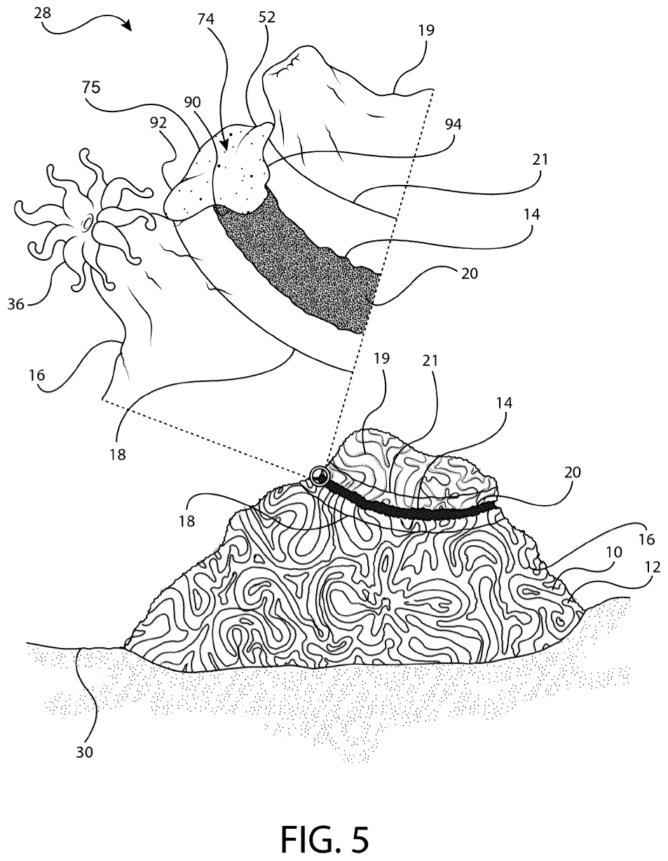

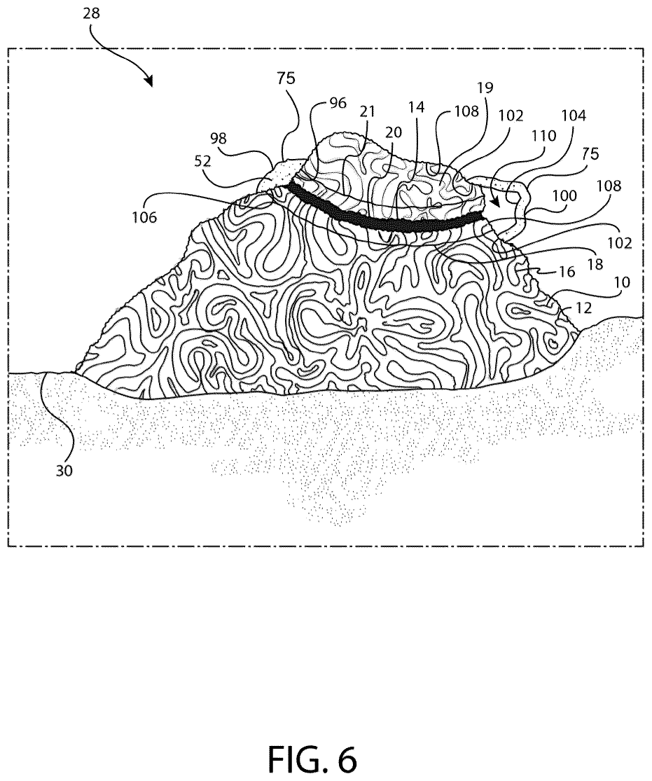

The at least one active agent 52 provides at least one important function to the first or second compositions 70, 72, namely providing an ingredient that can be dispensed or diffused to the target region 14 of the target body 10 (see the discussion of FIGS. 5 and 6 for more detail below) to provide a therapeutic effect. The delivery of the therapeutic effect from the at least one active agent 52 to the target region 14 that is part of the target body 10 for an extended period of time adds a lot of practical value to the composition body 75. In one embodiment, the composition body 75 can diffuse or dispense a therapeutically effective amount of the at least one active agent 52 to the target region 14 during the entire length of a diffusion period 74. See also the discussion of FIGS. 5-7 and 11 for a more detailed discussion about the diffusion period 74. The diffusion period 74 is the length of time that the composition body 75 can dispense or diffuse a therapeutically effective amount of the at least one active agent 52 to the designated target region 14 of the designated target body 10. In one embodiment, the diffusion period 74 is at least 12 hours, preferably at least 24 hours, more preferably at least 48 hours, or most preferably at least 72 hours. However, in at least one embodiment, the diffusion period may be as long as 1 month.

Still referring to FIG. 4, the therapeutic effect is not limited to delivering treatment. The therapeutic effect can be delivering an anti-microbial, antifungal, an antiviral, an antiprotozoal, an anti-bacterial, an antibiotic, an antiparasitic, an antiseptic, an anesthetic, a toxic, a quorum inhibiting, a nutrient providing, a quorum inhibiting, or an anti-inflammatory characteristic to the designated target region 14. In at least one embodiment, the toxic characteristic provided as the therapeutic effect is employed onto living barnacles that are secured to the submerged hull of a ship. In at least one embodiment, the application of ointment or composition body 75 itself creates a physical barrier that prevents or kills disease or infection which creates the beneficial effect. As an example, coral Black Band Disease is comprised of cyanobacteria which require oxygen to respirate that it usually obtains from the water so when the at least one base agent has a liquid impermeability characteristic, the composition is configured to treat black band disease by suffocating the cyanobacteria. Black Band Disease cyanobacteria also require light to photosynthesize so if the composition has a light diffusing or light blocking effect, the composition will be configured to kill the Black Band Disease by starving the cyanobacteria.

The ingredients that are suitable for the at least one active agent 52 include but are not limited to: Almond Extract, Aloe Vera Extract, Amla Extract, Anisyl Alcohol, Andrographolide, Amoxicillin Trihydrate, Antibiotics, Astragalus Extract, Azelaic Acid, Azithromycin, Baobab Fruit Extract, Barberry Extract, Basil Extract, Bayberry Extract, Bay Leaf Extract, Benzalkonium Chloride, Benzoic Acid, Benzyl Alcohol, Berberine, Bergamot Extract, Bilberry Extract, Black Pepper Extract, Black Seed Extract, Black Walnut Extract, Burdock Root Extract, Caffeine, Camphor Extract, Cannabinoids, Caprylyl Glycol, Cardamom Extract, Caryophyllene, Castor Oil, Cat Paw Extract, Cayenne Pepper Extract, Cedar Wood Extract, Cephalexin, Cetyl Palmitate, Chia Seed Extract, Chicory Root Extract, Chrysin, Clavulanate Potassium, Clindamycin, Cinnamon Extract, Ciprofloxacin, Clove Extract, Coconut Extract, Coriander Extract, Cranberry Extract, Curcumin, Dandelion Extract, Dragon's Blood Extract, Echinacea, Elderberry Extract, Ethanol, Ethylhexylglycerin, Eucalyptus Extract, Falcarinol, Fennel Seed Extract (Kaempferol), Ferulic Acid, Frankincense Extract, Gallic Acid, Garlic Extract, Ginger Extract, Gingergrass Extract, Ginseng Extract, Glyceryl Laurate, Glyceryl Undecylenate, Goldenseal Extract, Grape Seed Extract, Green Tea Extract, Grapefruit Extract, Hawthorn Extract, Honeysuckle Extract, Horseradish Extract, Humulene, Hydrogen Peroxide, lodine Solution, Isopropyl Alcohol, Juniper Extract, Lavender Extract, Lemon

Extract, Lemon Grass Extract, Lentil Extract, Lichen Extract, Licorice Extract, Maitake Mushroom Extract, Maltol, Manuka Honey Extract, Mandelic Acid, Milk Thistle Extract, Morinda citrifolia Extract, Mugwort Extract, Mullein Extract, Mustard Extract, Myricetin, Myrrh Extract, Naringenin, Naringin, Neem Extract, Nettle Extract, Ofloxacin, Onion Extract, Orange Extract, Oregano Extract, Pau d′Arco Extract, PEG-8-Laurate, Pentylene Glycol, Peppermint Extract, Phenethyl alcohol, Phenoxyethanol, Pinna Leaf Extract, Piperine, Polyquaternium-10, Polyquaternium-2, Polyquaternium-7, Polyquaternium-80, Polyphenols, Pomegranate Extract, Potassium Sorbate, Propanediol, Propolis, Pyrogallol, Quercetin, Quinine, Quinol, Qurecetin, Raspberry Extract, Red Clover Extract, Reishi Mushroom Extract, Resveratrol, Retinaldehyde, Rosemary Extract, Rutin, Sage Extract, Sagebrush Extract, Sandalwood Extract, Salicylic Acid, Sarsaparilla Extract, Sesame Extract, Shiitake Mushroom Extract, Sodium Benzoate, Sodium Sulfide, Sodium Bisulfide, Sodium Metabisulfite, Sodium Usnate, Sorbic Acid, Spearmint Extract, Strawberry Furanone/Furaneol, Sulfamethoxazole, Sunflower Extract, Syringic Acid, Tannic Acid, Tea Tree Extract, Theobromine, Thyme Extract, Turkey Tail Mushroom Extract, Turmeric Extract, Urea, Urea Peroxide, Ursolic Acid, Usnea Lichen Extract, Vanilla Extract, Vanillic Acid, Witch Hazel, Yarrow, and/or Zooxanthellae.

Still referring to FIG. 4, regarding the second formula 64 and the second composition 72, the at least one release modifier agent 68 is an ingredient that increases or decreases the rate of diffusion or dispensation of the at least one active agent 52 from the second composition 72. In certain embodiments, slowing down the speed that the at least one active agent 52 dispenses or diffuses from the second formula 64 will allow the therapeutic effect to be delivered for a longer period of time to the designated target region 14. In certain embodiments, increasing the speed that the at least one active agent 52 dispenses or diffuses from the second formula 64 will allow the therapeutic effect to be delivered for a shorter period of time to the designated target region 14 in a stronger dose to assure that a therapeutically effective amount of the active agent is delivered during the entire length of the desired diffusion period 74.

One of the most valuable attributes that the composition body 75 possesses is its viscoelastic behavior. The composition body is capable of application by means of constant repeatable extrusion to apply a uniform bead of material possessing extreme plasticity and distortion which is required to apply an unbroken bead of treatment across a variable surface of peaks and crevices present in aquatic animals such as corals. Additionally, the unique flow characteristics of the aquatic environment results in the need for the applied material to be capable of maintaining shape without succumbing to various flow and subsequent sheering effects of currents and waves.

To create a treatment capable of reliably delivering the above listed desired outcomes, an uncommonly high viscosity range was selected. As a general observation, most consumer materials including creams, pastes, and ointments do not target viscosity values above 250,000 centipoise regardless of viscosity testing method. It is conjectured that this is because the energy or force required to obtain materials from their packaging generally exceeds the average humans' capabilities as the viscosity climbs up to the range of 100,000 centipoise. At these large viscosity values, external mechanical forces are often required, and are achieved through the use of syringes, caulk type dispensers, or other dispensing devices.

Still referring to FIG. 4, to appropriately and consistently measure the material viscosities of the composition body 75 specific measuring techniques to measure the viscosity were established and chosen. To be clear, the measured viscosity of an object or material depends in part about how the viscosity itself is measured. The method consistently utilized by the inventors requires the use of a helipath in combination with T-bar spindles T-A through T-F, which corresponds to Brookfield viscometer entry codes 91-96. Measuring at 25° Celsius using the helipath combined with a T-bar spindle rotating in a range between 0 rpm to 5 rpm and measuring after 1 minute after the cross bar of the spindle enters the composition ensures unsheared material is constantly measured as the device apparatus helically rotates down, through the material. By constantly measuring unsheared material the viscoelastic behavior stays constant over time. Therefore, the selected method for measuring the viscosity of the desired composition body 75 is not subject to dynamic changes that may be a result of additional inherent characteristics of the material such as Thixotropic, Rheopectic, or Elastic behaviors.

A vital site of invention application is the Florida Reef Tract which extends from Martin County down through the Florida Keys. Depending on specific location, treatment depth, and season, the water temperatures may change as much as 10° Celsius with currents and wave action varying more. In response to this, the treatment material must also be dynamic which often requires viscosity modifications to meet the dynamic conditions at each site. Due to this a viscosity range of 500,000 centipoise to 50,000,000 centipoise specifically in correlation with the viscosity method mentioned above is targeted. This range ensures the compositions will be capable of proper extrusion and subsequent application throughout the many challenging environments it is intended to be utilized within.

To be clear, the measurement of the viscosity of the composition body 75 is measured utilizing Spindle Code: T-A through T-F (Entry code 91-96), running at a speed between 0 rpm and 5 rpm with the compositions body 75 temperature at 25 degrees Celsius. The measurement is taken one minute after the cross bar of the spindle enters the designated composition body 75.

Still referring to FIG. 4, using the above laid out measuring parameters, in at least one embodiment, the desired outcome of measured viscosity for the respective compositions body 75 is a first range between 500,000 Centipoise and 50,000,000 Centipoise. This first range is designed to allow sufficient material modification to ensure sufficient material extrusion and subsequent adhesion across all tropical regions, all seasons, and water conditions that many of the colonies of coral 12 on Earth reside within. Using the above laid out measuring parameters, in at least one other embodiment, the desired outcome of measured viscosity for the respective composition body 75 is between a second range of 750,000 Centipoise to 40,000,000 Centipoise. This second range is designed to permit more material modification to ensure enhanced material extrusion and subsequent enhanced adhesion across most tropical regions, during warm water seasons, in all boating conditions to the respective designated colonies of coral 12. Using the above laid out measuring parameters, in at least another embodiment, the desired outcome of measured viscosity for the respective composition body 75 is between a third range of 1,000,000 Centipoise and 30,000,000 Centipoise. This third range is designed to allow material modification to ensure best material extrusion and subsequent adhesion across some tropical regions, during warm water seasons, in favorable boating or aquatic conditions to the respective designated colonies of coral 12 or other target regions for applying the composition body 75.

Further, in other embodiments, the formulation 61 may include one or more additional ingredients 78, such as in the second formula 64 and third formula 65. The one or more additional ingredients 78 may include one or more of at least one efficacy enhancing agent 79, at least one pigment 80, at least one antioxidant/preservative 82, at least one quorum inhibiting agent 84, at least one taste deterrent 86, an astringent 88, or an adhesive agent 89. In one embodiment, the efficacy enhancing agent 79 increases or decreases the rate of diffusion of the at least one active agent 52 into the target region 14. Turning to a specific example, if the second formula 64 included an efficacy enhancing agent 79 and was applied to the designated target region 14 of an exemplary target body 10 of a designated colony of coral 12 such as shown in FIG. 1A, then the at least one release modifier agent 68 would increase or decrease the rate of diffusion of the at least one active agent 52 from the second formula 64, and the at least one efficacy enhancing agent 79 would increase or decrease the rate of diffusion of the at least one active agent 52 into the target region 14 of the colony of coral 12. In at least one embodiment, the at least one release modifier agent 68 affects the second formula 64, but the efficacy enhancing agent 79 influences the colony of coral 12 or other target regions 14 instead. In at least one other embodiment, the efficacy enhancing agent 79 acts upon the at least one active agent 52 directly to enhance the efficacy of the therapeutic properties of the at least one active agent 52 into the designated target region 14.

The additional ingredients 78 are considered optional to the first composition 70 and second composition 72 but at least one additional ingredient 78 is required to be present in the third composition 73. In addition, in at least one embodiment, the various additional ingredients 78 are integrally formed with either the at least one base agent 66 or the at least one active agent 52.

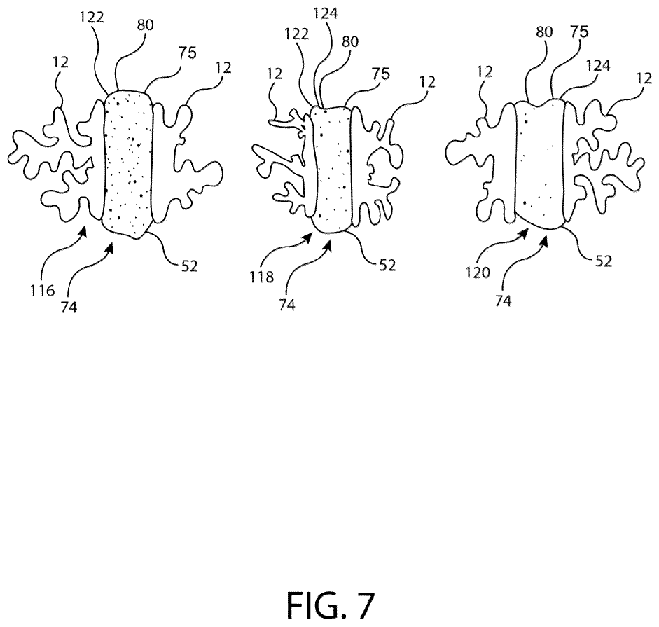

Still referring to FIG. 4, in another embodiment, the at least one pigment 80 is utilized to help indicate to an observer the amount of the at least one active agent 52 remaining in the respective formulation 61. See the discussion of FIG. 7 below for a more detailed discussion. In another embodiment, the at least one antioxidant/preservative 82 serves to help preserve the efficacy of the composition body 75 during storage prior to being applied to begin the diffusion period 74 or to assist preserving the at least one active agent 52 during the diffusion period 74.

In another embodiment, the at least one quorum inhibiting agent 84 is utilized to combat the mucosal response of bacteria or the colony of coral 12. Bacteria or the colony of coral 12 can produce mucous in response to the presence of a “foreign body” such as the designated treatment composition body 75 being applied into their environment. That is, in at least one embodiment, the at least one quorum inhibiting agent 84 in one of the compositions 75 can help ensure that bacteria present on the surface of the designated colony of coral 12 or the colony of coral 12 itself do not produce as much mucous. This stops the bacteria or the colony of coral 12 from blocking out the designated composition body 75 from diffusing the at least one active agent 52 to the target region 14 of the target body 10.

Additionally, the at least one quorum inhibiting agent 84 helps to prevent or retard any bacteria or other mucous producing organisms from forming and organizing a biofilm barrier to block out a foreign body in the local environment, such as when one of the designated composition body 75 is applied to the target region 14 of the designated target body 10 of a designated colony of coral 12. At least some kind of bacteria can operate in an organized fashion to form a biofilm barrier to try to protect the remainder of the colony of bacteria from an external threat, such as an application the composition body 75. This organized response by the colony of bacteria is organized with a series of chemical signals to mobilize the local bacteria to form a bacteria barrier, or biofilm. The presence of a biofilm forming between the designated compositions body 75 and the target region 14 could retard or prevent the diffusion of the at least one active agent 52 from diffusing successfully to the target region 14. The at least one quorum inhibiting agent 84 prevents or retards that communication between the members of the colony of bacteria. Absent that communication, the organized barrier or biofilm can be retarded or prevented from forming altogether.

Still referring to FIG. 4, in other embodiments, the at least one astringent 88 can bind mucosal proteins, retarding their ability to create an effective barrier. Therefore, the addition of the at least one astringent 88 can help control or at least stop the prevention or retardation of the diffusion of the at least one active agent 52 during the diffusion period 74 to the designated target region 14. Please see the discussion of FIGS. 8 and 9 for a more detailed discussion of the at least one quorum inhibiting agent 84, and the at least one astringent 88.

Still referring to FIG. 4, in other embodiments, the at least one adhesive agent 89 helps ensure composition body 75 is capable of adhering or cohering to the intended target for the necessary time periods. Note, within this disclosure “adhesion” or “adhesive effects” refer specifically to submerged aquatic adhesion. This often includes ingredients that are typically not considered to be adhesive agents in dry environments.

In other embodiments, a taste deterrent 86 is added to dissuade fish and other marine animals from feasting on the applied composition body 75 before the diffusion or dispensation of the at least one active agent 52 is complete. For instance, when the at least one active ingredient is algae or other nutrients, marine animals may begin to eat the composition 61 before dispensation is complete. By adding the taste deterrent 86 to the respective composition 61, the composition body 75 will be able to dispense or diffuse the at least one active agents 52 properly on a more consistent basis.

The additional ingredients 78 can be mixed into the designated formulation 61 at the same time when the respective composition body 75 is formed, or the additional ingredients 78 can be added before the formation of the respective composition body 75 or after. However, the additional ingredients 78 will only be deemed to be part of the composition body 75 if the additional ingredients 78 are present in the designated composition body 75 prior to the initiation of the diffusion period 74 for the designated composition body 75 at the designated target region 14.

In other embodiments, the active agent 52 can be mixed into the designated formulation 61 at the same time as the respective composition body 75 is formed or they may be added by the end user at a later time. However, the active agent 52 must be present in the designated composition body 75 prior to the initiation of the diffusion period 74 at the designated target region 14 to be considered part of the composition body 75.

In another embodiment, the active agent 52 is not present but the at least one base agent 66 and at least one of the additional ingredients 78 which comprise the formulation 65 are combined to form the third composition 73. Third composition 73 may act as a physical barrier to prevent infection of the target body 10 or may act as a benefit to the target body 10 by blocking light, water, air, bacteria, or viruses from entering the target region 14 due to the beneficial characteristics of the composition body 75. For example, the third composition 73 may serve as a way to block light to a Black Band Disease infection to stop photosynthesis, or may retard water permeability to cut off the oxygen supply to a Black Band Disease infection in the covered target region 14.

Referring to FIG. 5, another exemplary colony of coral 12 serves as the target body 10 with the target region 14 bounded by the first target boundary 18 which demarcates the target region 14 from the first non-targeted region 16, and the second target boundary 21 which demarcates the target region 14 from the second non-targeted region 19 between which contains the black band 20. A portion of the colony of coral 12 located near the first target boundary 18 has been expanded to illustrate one of the healthy polyps 36 of the colony of coral 12 being separated from one of the damaged, injured or dead portions of the colony of coral 12, namely the second non-targeted region 19, located on the other side of the black band 20. An application of the composition body 75 (in this FIG. the first composition body 70) has been applied onto a portion of the first target boundary 18, the second target boundary 21, and the target region 14. To be clear, there is often no harm from applying the composition body 75 to portions of the first non-targeted region 16 and the second non-targeted region 19, and the composition body 75 can diffuse through those respective regions 16, 19 into the target region 14. But the delivery of the active agent 52 is more effective when applied to the target region 14, and also avoids any chance of harming the healthy colonies of coral 12 inadvertently by forming a barrier between the healthy coral of the first non-targeted region 16 and the liquid environment 28. To be even clearer, the overlap of the composition body 75 over the first non-targeted region 16 and the second non-targeted region 19 has been exaggerated in the drawing to illustrate that, in at least one embodiment, some of the composition body 75 is not directly in contact with the target region 14. The composition body 75 has a bottom or first surface 90 and a top or second surface 92. The target region 14 has an upper or third surface 94 that contacts the first surface 90. The second surface 92 does not contact the target body 10, the target region 14 or the first non-targeted region 16, but does contact the liquid environment 28. When the composition body 75 is applied to the target region 14, the composition body 75 begins a diffusion period 74 when the therapeutically effective amount of the at least one active agent 52 is diffused or dispensed to the target region 14 over the length of the diffusion period 74 through the first surface 90.

Once again, as discussed above, the therapeutically effective amount of the at least one active agent 52 is dispensed or diffused to the target region 14 during the entire length of the diffusion period 74. This allows the designated composition body 75 to dispense an effective amount of treatment to the designated target body 10 without requiring re-capture or re-application over a substantial period of time for the designated target body 10 that is located in the liquid environment 28 at a minimum during high tide 32, and more often than not, submerged continuously within the liquid environment 28 during the length of the entire diffusion period 74. This saves a great deal of time, reduces stress on the living target organism(s) (if they are alive), and makes a single application of one of the designated composition bodies 75 a more cost-effective solution for treating aquatic animals, plants and non-organic target bodies located in the designated liquid environment 28 than previous strategies.

Referring to FIG. 6, a different exemplary colony of coral 12 that serves as the target body 10 with the target region 14 separated from the first non-targeted region 16 by the first target boundary 18 located near the black band 20 and separated from the second non-targeted region 19 by the second target boundary 21 is shown. Two applications of the composition body 75 are shown in cross section, one generally off to the left and one off to the right when viewing FIG. 6. The target body 10 is positioned on the floor 30. The composition body 75 on the left side has a lower or fourth surface 96 and a higher or fifth surface 98, the composition body 75 on the right side has an outer or sixth surface 100, a connecting or seventh surface 102, and an inner or eighth surface 104, and the target region 14 has a left or ninth surface 106 and a right or tenth surface 108 as shown. The fourth surface 96 of the composition body 75 on the left side is in direct contact with the ninth surface 106 of the target region 14. The tenth surface 108 of the target region 14 contacts an interstitial space or first volume of liquid 110 that is in contact with the eighth surface 104 of composition body 75 on the right side of FIG. 6. In one embodiment, this first volume of liquid 110 is in direct contact with the liquid environment 28. In this embodiment, as shown in FIG. 6, this first volume of liquid 110 is not in direct contact with the liquid environment 28. That is, the composition body 75 on the right side of FIG. 6 is between the liquid environment 28 and the first volume of liquid 110. The first volume of liquid 110 may have as little as 10% water by weight as a percentage of the weight of the first volume of liquid 110, as opposed to the higher water content percent required to be present in the liquid environment 28.

The direct contact of the composition body 75 (through the fourth surface 96) on the left to the surface of the target region 14 (through the ninth surface 106) allows and permits the direct diffusion or dispensation of the at least one active agent 52 from the composition body 75 on the left to the target region 14. However, the tenth surface 108 of the target region 14 is only in indirect contact with the eighth surface 104 of the composition body 75 on the right side of FIG. 6 due to the presence of the first volume of liquid 110 located between the composition body 75 on the right and the target region 14. The seventh surface 102 does not contact the target region 14 directly, and has a relatively small surface area as shown. The composition body 75 on the right can still effectively dispense or diffuse the at least one active agent 52 to the target region 14 if the therapeutically effective amount of the at least one active agent 52 diffuses or dispenses to the target region 14, whether the contact is direct or indirect contact. The fifth surface 98 and the sixth surface 100 of the left and right side composition body 75 both contact the surrounding liquid environment 28. That is, when the at least one active agent 52 diffuses through the first volume of liquid 110 when the composition body 75 is in a first position 112 (see FIG. 6) and diffuses the at least one active agent 52 from the composition body to the target region 14 through the first volume of liquid 110.

In another embodiment, the designated composition body 75 can have a layer of protecting material (not shown) such as a bandage, film, plastic, polymer or water impermeable material applied to a portion of their respective surfaces, such as the fifth surface 98 and the sixth surface 100 of the composition body 75 illustrated in FIG. 6. The layer of protecting material serves as a way to reduce the diffusion and interaction between the respective composition body 75 and the liquid environment 28. For instance, the protecting material layer can be deployed to reduce the amount of erosion of the respective composition bodies from ocean currents or exposure to an air environment during low tide. Not to be constrained by theory, it is believed that the layer of protecting material can help the respective composition body 75 diffuse or dispense the at least one active agent 52 for a longer period of time when present in many applications and embodiments.

FIG. 7 illustrates an exemplary composition body 75 that has at least one pigment 80 and is applied to another colony of coral 12 during a first period of time 116 during a designated diffusion period 74, a second period of time 118 during the same designated diffusion period 74, and a third period of time 120 during the same designated diffusion period 74. The first period of time 116 is earlier in time than the second period of time 118, which is earlier in time than the third period of time 120. The composition body 75 has a first color 122 during the first period of time 116, and a second color 124 during the third period of time 120, and a combination of the first color 122 and the second color 124 during the second period of time 118. When the at least one pigment 80 is present in a higher percentage, such as directly before the application of the composition body 75 prior to the diffusion period 74, the composition body 75 tends to exhibit the first color 122. When the at least one pigment 80 is present in a lower percentage in the composition body 75, such as near the end of the associated diffusion period 74, such as during the third period of time 120, the composition body 75 tends to exhibit the second color 124.

In the second period of time 118, which is between the first period of time 116 and the third period of time 120, the color exhibited by the composition body 75 is a combination of the first color 122 and the second color 124. The percentage of the at least one pigment 80 reduces as more and more of the at least one pigment 80 is diffused from the composition body 75. In this way, in at least one embodiment, the change of the exhibited color of the designated composition body 75 from the first color 122 to the second color 124 positively correlates with the diffusion of both the at least one pigment 80 and the at least one active agent 52 from the designated composition body 75. In other words, the change in color in the composition body 75 from the first color 122 toward the second color 124 serves as an indication (i.e. there is a positive correlation to) of the amount of the at least one active agent 52 that has been diffused or dispensed from the designated composition body 75. In this way, an observer has a simple but effective way to decide when the diffusion period 74 is effectively over, and more applications of the composition body 75 can be administered, if necessary.

Still referring to FIG. 7, in at least one embodiment, the at least one active agent 52 of the designated composition body 75 has a first weight at the beginning of the designated diffusion period 74 and the composition body 75 diffuses or dispenses at least 25%, preferably at least 40% of the first weight of the at least one active agent 52 during the length of the associated diffusion period 74. In at least one embodiment, the at least one pigment 80 displays a color wavelength to observers within a range of about 380 nanometers to about 590 nanometers (nm), preferably within a range of 380 nm to 520 nm, and more preferably within a range of 380 nm to 445 nm.

Referring to FIG. 8, both coral polyps and/or exemplary colony of bacteria 126 respond to the presence of an exemplary application of one of the designated compositional bodies 75 over time when none of the at least one astringent 88, and none of the at least one quorum inhibiting agent 84 are present to interfere with the response by the colony of bacteria 126. In the first time interval 128, the designated composition body 75 is in direct contact with the colony of bacteria 126. In at least one embodiment, the colony of bacteria 126 sends a signal to the colony to fight back against a hostile foreign object, namely the designated composition body 75. In the first reaction 130 that occurs before a second time interval 132 which occurs after the first time interval 128, the colony of bacteria 126 expands and begins to form a biofilm 134. In the second reaction 136 that occurs before a third time interval 140 which occurs after the second time interval 132, the volume of the colony of bacteria 126 expands more and more, thereby lifting the designated composition body 75. In a third reaction 142 that occurs before a fourth time interval 144 which occurs after the third time interval 140, the volume of the colony of bacteria 126 expands and changes shape to push the designated composition body 75 into a less flat shape.

In a fourth reaction 145 that occurs before a fifth time interval 146 which occurs after the fourth time interval 144, the volume of the colony of bacteria 126 expands and changes shape to push the designated composition body 75 into an even less flat shape, and a first portion 148 of the colony of bacteria 126 starts to separate or pinch away from a second portion 150 of the colony of bacteria 126. This pinching and separating action tends to protect the second portion 150 of the colony of bacteria 126 from the designated composition body 75. In a fifth reaction 152 that occurs before a sixth time interval 154 which occurs after the fifth time interval 146, the volume of the colony of bacteria 126 expands and changes shape to break off the first portion 148 of the colony of bacteria 126 from the second portion 150 of the colony of bacteria 126. The designated composition body 75 floats away into the liquid environment 28 along with the first portion 148, and the second portion 150 of the colony of bacteria 126 is left undisturbed, and the application of the designated composition body 75 is rendered ineffective.

FIG. 9 depicts an exemplary colony of bacteria 126 reacting to the presence of a designated composition body 75 by attempting to initiate a mucosal response. The composition body 75 includes at least one of the two specific additional ingredients 78, namely the at least one quorum inhibiting agent 84, and/or the at least one astringent 88. It is noteworthy that the colony of coral 12 itself can also try to form a mucosal response upon exposure of a foreign body, such as the application of the composition body 75. In a beginning time interval 156 the designated composition body 75 is in direct contact with the colony of bacteria 126. During an initial reaction 158 which occurs before a middle time interval 160 the colony of bacteria 126 begins to shrink due to the presence of at least one of the two additional ingredients 78 listed above. In some embodiments, the at least quorum inhibiting agent 84 prevents the colony of bacteria 126 from successfully forming biofilm (not shown) as a form of protection. In FIG. 9, the production of mucous is interrupted instead, as shown in the detailed cross section labeled 161. In some embodiments, the colony of bacteria 126 loses volume as the at least one astringent 88 attacks the colony of bacteria 126 directly. During a later reaction 162 which occurs after the middle time interval 160 and before a later time interval 164, the colony of bacteria 126 has substantially shrunk in size, and is not serving as an effective barrier to the dispensation or diffusion of the at least one active agent 52 from the designated composition body 75 to any underlying tissue or target region 14 opposite the original colony of bacteria 126.

As best shown in the detailed cross section 161, the at least astringent 88 form nuclei 163 that attract a collection of mucosal proteins 165 that would otherwise expand the volume of the bacteria 126 which would otherwise form an effective mucosal response. In other words, the at least one astringent 88 improves the adhesion of composition to the target body 12 and/or diffusion of the at least one active agent 52 to the target region 14 by binding or clumping either bacterial mucosal proteins and/or target body mucosal proteins.

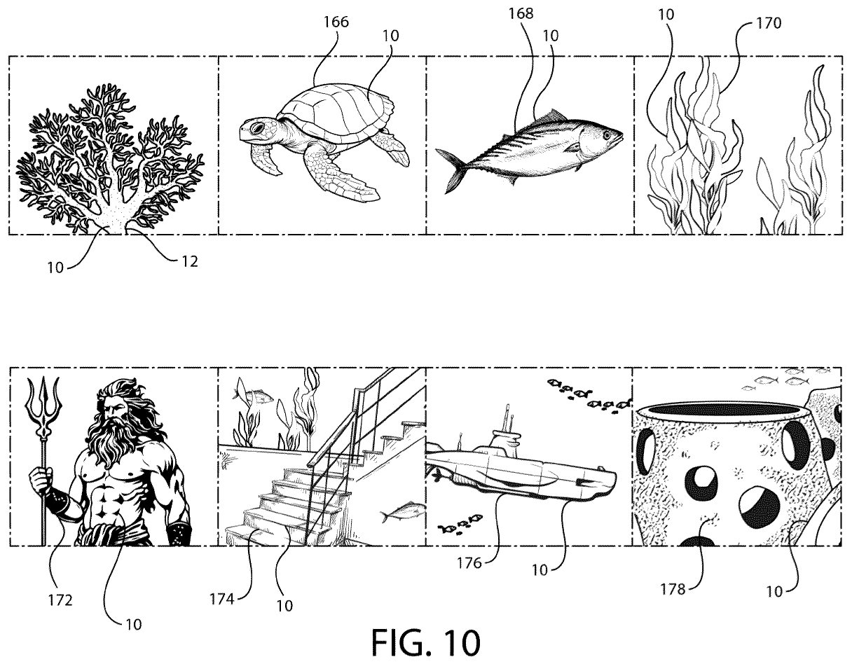

FIG. 10 depicts images of a variety of potential contemplated organic target bodies 10 and non-organic target bodies 10. Exemplary organic target bodies 10 include a designated colony of coral 12, a sea turtle 166, a fish 168, and at least one sea kelp 170. Exemplary non-organic target bodies 10 include a statue 172, a submerged set of stairs 174, a submarine hull 176, a shell produced by a now-deceased barnacle (not shown), and a manmade reef module 178. In other embodiments, the contemplated target body 10 is at least one coral polyp 36 (see FIG. 3), sharks 26 (see FIG. 1D), a reptile such as an alligator (not shown), an amphibian such as a frog (not shown) and manatees 22 (see FIG. 1C). In other embodiments, the contemplated target body 10 can be a barnacle shell (not shown), a pane of glass (not shown) such as a glass wall of a small aquarium, a propeller (not shown), or a boat hull (not shown). In some embodiments, the contemplated target region 14 of the designated target body 10 may be a lesion site (not shown), wound sites 24 (see FIGS. 1C and 1D), diseased sites 14 (see FIGS. 1A and 1B), an infection site (not shown, but see the diseased sites for FIGS. 1A and 1B), and one or more coral polyps 36 (see at least FIGS. 1A, 1B, and 5).

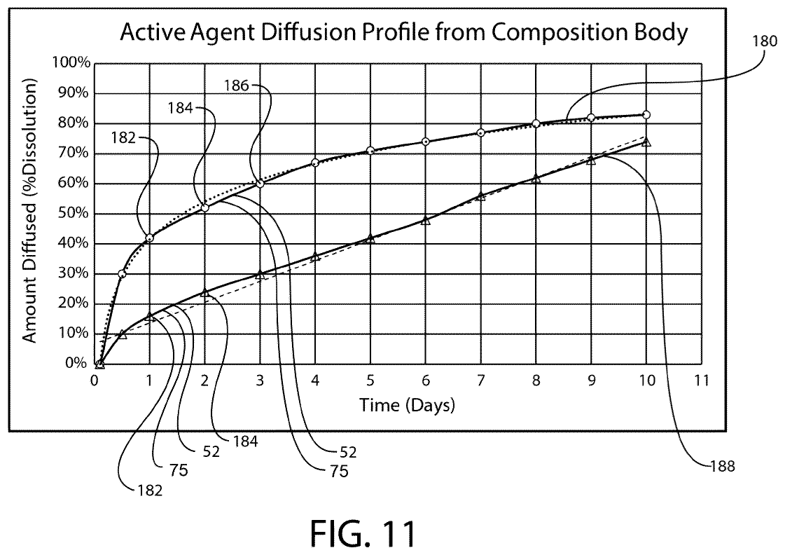

FIG. 11 is a reproduction of a graph that measures time in days on the “X” axis and measures the amount of a representative at least one active agent 52 that has been diffused or dispensed from two representative composition bodies 75 as a percentage on the “Y” axis to their respective representative target regions 14. A first dispensation curve 180 (the higher one in FIG. 11) shows that about 40% of the at least one active agent 52 was diffused or dispensed by “day 1” in a first day interval 182, then diffused or dispensed a total of 50% of the at least one active agent 52 by “day 2” in a second day interval 184, and diffused or dispensed a total of 60% of the at least one active agent 52 by “day 3” in a third day interval 186. This means that the representative composition body 75 represented by the first dispensation curve 180 dispensed 40% of the at least one active agent 52 on day 1, then dispensed 10% more on day 2, and 10% more on day 3. The first representative composition body 75 dispensed 80% of the at least one active agent 52 by “day 10”.

A second dispensation curve 188 (the lower one in FIG. 11) shows that about 15% of the at least one active agent 52 was diffused or dispensed by “day 1” in the first day interval 182, then diffused or dispensed a total of 25% of the at least one active agent 52 by “day 2” in the second day interval 184, and diffused or dispensed a total of 30% of the at least one active agent 52 by “day 3” in the third day interval 186. This means that the representative composition body 75 represented by the second dispensation curve 188 (the lower one) dispensed 15% of the at least one active agent 52 on day 1, then dispensed 10% more on day 2, and 5% more on day 3. The second representative composition body 75 dispensed about 70% of the at least one active agent 52 by “day 10”.

The graph depicted in FIG. 11 illustrates that the at least one active agent 52 diffuses or dispenses to the target region 14 at a first rate during a first time interval, at a second rate during a second time interval, and at a third rate during a third time interval, wherein the first rate is greater than the second rate when the first time interval is before than the second time interval, and the second rate is greater than the third rate when the second time interval is before the third time interval. Further, the aforementioned first rate, second rate, and third rate form a logarithmic shaped curve when time elapsed is measured versus the rate of diffusion or dispensation to the target region 14.

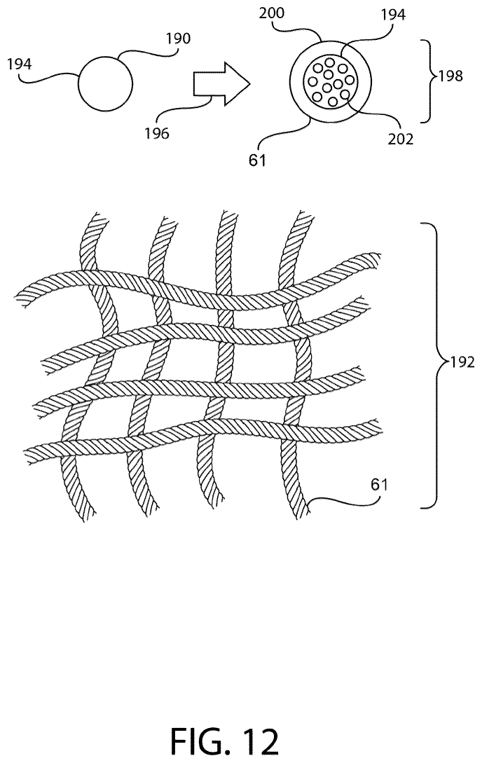

FIG. 12 illustrates an absorbent fiber or absorbent medium 190 that is transformed ultimately into an impregnated woven sheet 192. The absorbent medium 190 may be made of organic or non-organic fibers or threads, such as jute, bamboo, nylon, recycled plastic, etc., and has an exterior surface 194. The absorbent medium 190 is soaked, mixed, or dipped with a representative formulation 61. This act of soaking, mixing or dipping collectively will be described herein as a soaking process 196. After the absorbent medium 190 is subjected to the soaking process 196, the absorbent medium 190 transforms into a coated or impregnated medium 198. The impregnated medium 198 may have an outer coating 200 of the representative formulation 61 exterior of the exterior surface 194, or may have an internal impregnation 202 of the representative composition 61 inside the confines of the exterior surface 194, or both. When several of the impregnated mediums 198 are woven together, they form the impregnated woven sheet 192. In certain embodiments, the impregnated woven sheet 192 can be a convenient way to deliver the formulation 61 to the target region 14 by securing the impregnated woven sheet 192 to be in direct contact with the target region 14 or to be separated from the target region 14 by a representative first volume of liquid 110 (see the discussion of FIG. 6 above for a more detailed discussion). In this way, the formulation 61 can be prepared in a way to reduce the amount of time required within the liquid environment 28 to apply the formulation 61 to the designated target regions 14.

What is claimed is:

1. A method of using a composition suitable for use in a liquid environment to convey a therapeutic effect comprising:
providing at least one base agent having a hydrophobic property and at least one beneficial characteristic;
providing at least one active agent having at least one therapeutic property;
providing a target body having a target region;
providing a liquid environment comprising at least 75% water;
combining the at least one base agent and the at least one active agent to form a formulation that is mixed evenly to form a first composition body;
applying the first composition body to the target region for at least a period of time defining a diffusion period;
wherein the first composition body diffuses a therapeutically effective amount of the at least one active agent which conveys the at least one therapeutic property to the target region during the diffusion period,
wherein the first composition body has a viscosity between a range of 500,000 to 50,000,000 centipoise, preferably a range of 750,000 to 40,000,000 centipoise, or more preferably a range of 1,000,000 to 30,000,000 centipoise, when the viscosity is measured by heating the first composition body to 25 degrees Celsius, using a T Bar Spindle (A-F) with helipath and taking the viscosity reading after rotating the first composition body between 0 rotations per minute to 5 rotations per minute at 1 minute after the cross bar of the spindle enters the first composition body,
wherein the first composition body has an adhesive property, and
wherein the target region is submerged in the liquid environment continuously during the diffusion period or is submerged in the liquid environment at high tide as a result of the liquid environment being subject to tidal forces.

2. The method of using the composition of claim 1, wherein the liquid environment comprises at least 80% water, preferably at least 85% water, or more preferably at least 95% water, and wherein the first composition body is in contact with the target region during the entire length of the diffusion period.

3. The method of using the composition of claim 1, wherein the first composition body diffuses a therapeutic effective amount of the at least one active agent to the target region, wherein the diffusion period is at least 12 hours, preferably at least 24 hours, more preferably at least 48 hours, or most preferably at least 72 hours.

4. The method of using the composition of claim 1, wherein the at least one therapeutic property has an antimicrobial, an antifungal, an antiviral, an antiprotozoal, an antiparasitic, an antiseptic, an anesthetic, a toxic, a nutrient providing, a quorum inhibiting, an algae providing, or an anti-inflammatory characteristic.

5. The method of using the composition of claim 4, wherein the at least one therapeutic property is conveyed to an aquatic organism belonging to Class Anthozoa, Malacostraca, Amphibia, Bivalvia, Cephalopoda, Gastropoda, Marine Based Mammalia, Reptilia, or Superclass Osteichthyes.

6. The method of using the composition of claim 4, wherein the at least one therapeutic property is conveyed to an aquatic organism belonging to Class Anthozoa, Amphibia, Cephalopoda, Gastropoda, Marine Based Mammalia, or Reptilia.

7. The method of using the composition of claim 6, wherein the first composition body diffuses a therapeutically effective amount of the at least one active agent to the target region, wherein the diffusion period is at least 12 hours, preferably at least 24 hours, more preferably at least 48 hours, or most preferably at least 72 hours.

8. The method of using the composition of claim 7, wherein the diffusion period conveys the at least one therapeutic property to the target region from the at least one active ingredient at a first rate during a first time interval, at a second rate during a second time interval, and at a third rate during a third time interval, wherein the first rate is greater than the second rate when the first time interval is before the second time interval, and the second rate is greater than the third rate when the second time interval is before the third time interval.

9. The method of using the composition of claim 1, wherein the at least one beneficial characteristic is air impermeability, air permeable retardation, liquid impermeability, liquid permeable retardation, a light diffusing effect, a light blocking effect, a viral impermeability, a viral permeable retardation, a bacterial impermeability, or a bacterial permeable retardation.

10. The method of using the composition of claim 1, wherein the composition further includes at least one taste deterrent that is configured to discourage aquatic life such as fish from eating the first composition body, at least one antioxidant/preservative, at least one astringent, at least one pigment, at least one efficacy enhancing agent, or at least one adhesive agent.

11. A method of using a composition suitable for use in a liquid environment having a beneficial effect comprising:
providing at least one base agent having a hydrophobic property and at least one beneficial characteristic;
providing a target body having a target region;
providing a liquid environment comprising at least 75% water;
mixing evenly a formulation that comprises the at least one base agent to form a first composition body having an adhesive property;
applying the first composition body to the target region for at least a period of time defining a first interval of time;
wherein the first composition body has a viscosity between a range of 500,000 to 500,000,000 centipoise, preferably a range of 750,000 to 40,000,000 centipoise, more preferably a range of 1,000,000 to 30,000,000 centipoise, when the viscosity is measured by heating the first composition to 25 degrees Celsius, using a T Bar Spindle (A-F) with helipath and taking the viscosity reading after rotating the first composition between 0 rotations per minute to 5 rotations per minute at 1 minute after the cross bar of the spindle enters the first composition body, and
wherein the target region is submerged in the liquid environment continuously or is submerged in the liquid environment at high tide as a result of the liquid environment being subject to tidal forces during the first interval of time.

12. The method of using the composition of claim 11 wherein the liquid environment comprises at least 80% water, preferably at least 85% water, or more preferably at least 95% water, and wherein the first composition body is in contact with the target region during the entire length of the first interval of time.

13. The method of using the composition of claim 11 wherein the first interval of time is at least 12 hours, preferably at least 24 hours, more preferably at least 48 hours, or most preferably at least 72 hours.

14. The method of using the composition of claim 11 wherein the target region is located on a surface of an aquatic organism belonging to Class Anthozoa, Malacostraca, Amphibia, Cephalopoda, Gastropoda, Marine Based Mammalia, Reptilia, or Superclass Osteichthyes.

15. The method of using the composition of claim 13, wherein the target region is located on a surface of an aquatic organism belonging to Class Anthozoa, Amphibia, Bivalvia, Cephalopoda, Gastropoda, Marine Based Mammalia, or Reptilia.

16. The method of using the composition of claim 11, wherein the at least one beneficial characteristic is air impermeability, air permeable retardation, liquid impermeability, liquid permeable retardation, a light diffusing effect, a light blocking effect, a viral impermeability, a viral permeable retardation, a bacterial impermeability, or a bacterial permeable retardation.

17. The method of using the composition of claim 11, wherein the composition further includes at least one taste deterrent that is configured to discourage aquatic life such as fish from eating the first composition body, at least one antioxidant/preservative, at least one astringent, at least one pigment, at least one efficacy enhancing agent, or at least one adhesive agent.

18. The method of using the composition of claim 1, wherein the method of using the composition includes a further step of providing at least one release modifier, wherein the formulation of the first body composition further comprises the at least one release modifier agent.

19. The method of using the composition of claim 18, wherein the at least one release modifier agent slows or accelerates the conveying of the at least one therapeutic property of the at least one base agent to the target region during the diffusion period.

20. A method of using a composition suitable for use in a liquid environment having a beneficial effect comprising:
providing at least one base agent having a hydrophobic property and at least one beneficial characteristic;
providing a target body having a target region;
providing a liquid environment comprising at least 75% water;
Mixing evenly a formulation that comprises the at least one base agent to form a first composition body having an adhesive property;
applying the first composition body to the target region for at least a period of time defining a first interval of time;
wherein the first composition body has a viscosity between a range of 500,000 to 500,000,000 centipoise, preferably a range of 750,000 to 40,000,000 centipoise, more preferably a range of 1,000,000 to 30,000,000 centipoise, when the viscosity is measured by heating the first composition to 25 degrees Celsius, using a T Bar Spindle (A-F) with helipath and taking the viscosity reading after rotating the first composition between 0 rotations per minute to 5 rotations per minute at 1 minute after the cross bar of the spindle enters the first composition body, and
wherein the target region is submerged in the liquid environment continuously or is submerged in the liquid environment at high tide as a result of the liquid environment being subject to tidal forces during the first interval of time, and
wherein the at least one therapeutic property is conveyed to an inorganic aquatically located substrate such as a set of stairs, a reef module, a shell produced by a barnacle, a statue, a pane of glass, a pane of acrylic, a propeller, an aquarium, or a boat hull.
